(19) United States
(12) Patent Application Publication
(10) Pub. No.: US 2025/0332344 A1
(43) Pub. Date: Oct. 30, 2025

(54) System, Method, and Computer Program Product for Detecting Fluid Flow Through Ultrasonic Flow Sensors and/or Identifying Medication Diversion

(71) Applicant: Becton, Dickinson and Company, Franklin Lakes, NJ (US)

(72) Inventor: Amir Mohammad Tahmasebi Maraghoosh, Newton, MA (US)

(21) Appl. No.: 18/650,344

(22) Filed: Apr. 30, 2024

(51) Int. Cl. A61M 5/168; G16H 20/17

(52) U.S. Cl. CPC A61M 5/16886; G16H 20/17

(57) ABSTRACT

Systems, methods, and computer program products are provided for detecting fluid flow through ultrasonic flow sensors and/or identifying medication diversion. An example system includes at least one processor configured to: generate, using at least one machine learning model, based on a time-series generated by an ultrasonic flow sensor, a predicted start time associated with a start of a flow, a predicted time period associated with the flow, and/or a predicted end time associated with an end of the flow; and/or identify, based on the time-series, in a database, at least one stored feature representation associated with at least one medication type.

### BACKGROUND

### Technical Field

This disclosure relates generally to ultrasonic flow sensors and, in non-limiting embodiments or aspects, to systems, methods, and computer program products for detecting fluid flow through ultrasonic flow sensors and/or identifying medication diversion.

### Technical Considerations

Existing methodology for detecting a start and an end of a medication administration via an ultrasonic flow sensor may rely on shift detection in up and down stream signals generated by two ultrasound crystals at respective ends of a flow tube of the ultrasonic sensor and defining a specific threshold to determine the start and end of the medication administration, which is sensitive to the signal-to-noise ratio issues, as well as the hard-coded threshold value, which may not necessarily be valid for all types of medications.

Existing methodology for identifying medication diversion at a time of administration may rely on reading a barcode on printed on a label and attached to a syringe. However, existing methodology cannot verify a match between medication inside the syringe or delivered via a flow sensor and the medication identified by the label on the syringe.

### SUMMARY

Accordingly, provided are improved systems, methods, and computer program products for detecting fluid flow through ultrasonic flow sensors and/or identifying medication diversion.

According to non-limiting embodiments or aspects, provided is a system, including: at least one processor configured to: receive a time-series generated by an ultrasonic flow sensor corresponding to a flow of at least one fluid through a fluid flow path of the ultrasonic flow sensor; generate, using at least one machine learning model, based on the time-series, at least one of (i) a predicted start time associated with a start of the flow of the at least one fluid through the fluid flow path of the ultrasonic flow sensor, (ii) a predicted time period associated with the flow of the at least one fluid through the fluid flow path of the ultrasonic flow sensor, (iii) a predicted end time associated with an end of the flow of the at least one fluid through the fluid flow path of the ultrasonic flow sensor, or any combination thereof; and provide the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the end predicted time associated with the end of the flow of the at least one fluid through the fluid flow path of the ultrasonic flow sensor, or any combination thereof.

In some non-limiting embodiments or aspects, the time-series includes at least one of a plurality of amplitudes at a plurality of time points, a plurality of phases at the plurality of time points, or any combination thereof, and wherein the at least one processor is further configured to: generate, using at least one feature extraction algorithm, based on the time-series including the plurality of amplitudes at the plurality of time points, the plurality of phases at the plurality of time points, or any combination thereof, a feature representation associated with the time-series, and wherein the at least one processor is configured to generate, using the at least one machine learning model, based on the time-series, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, by: providing, as input to the at least one machine learning model, the feature representation associated with the time-series; and receiving, as output from the at least one machine learning model, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof.

In some non-limiting embodiments or aspects, the at least one processor is further configured to: identify, based on the time series generated by the ultrasonic flow sensor corresponding to the flow of the at least one fluid through the fluid flow path of the ultrasonic flow sensor, in a database including a plurality of stored feature representations associated with a plurality of medication types, at least one stored feature representation of the plurality of stored feature representations, wherein the at least one stored feature representation is associated with at least one medication type; and provide an indication of the at least one medication type.

In some non-limiting embodiments or aspects, the at least one processor is further configured to: obtain at least one expected medication type; determine whether the at least one expected medication type corresponds to the at least one medication type associated with the identified at least one stored feature representation; and provide an indication of whether the at least one expected medication type corresponds to the at least one medication type associated with the identified at least one stored feature representation.

In some non-limiting embodiments or aspects, the at least one processor is configured to generate, using the at least one machine learning model, based on the time-series and at least one of the at least one expected medication type, the at least one medication type, or any combination thereof, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof.

In some non-limiting embodiments or aspects, the at least one processor is further configured to: determine, based on the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, at least one of an amount of the at least one fluid that flowed through the fluid flow path of the ultrasonic flow sensor, a rate at which the at least one fluid flowed or is flowing through the fluid flow path of the ultrasonic flow sensor, or any combination thereof; and provide the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof.

In some non-limiting embodiments or aspects, the at least one processor is further configured to: automatically store, in a database, in association with a record of a patient, the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof.

In some non-limiting embodiments or aspects, the at least one processor is further configured to: determine whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates at least one of a threshold amount associated with at least one fluid, a threshold rate associated with the at least one fluid, or any combination thereof; and provide an indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof.

In some non-limiting embodiments or aspects, the at least one processor is configured to provide the indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof, by controlling a user interface associated with the ultrasonic flow sensor to output at least one of an audio output, a visual output, or any combination thereof associated with whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof.

In some non-limiting embodiments or aspects, the system further includes: the ultrasonic flow sensor, wherein the ultrasonic flow sensor includes a flow tube that defines the fluid flow path of the ultrasonic flow sensor, a first piezoelectric sensor or transducer arranged at an upstream position of the flow tube, and a second piezoelectric sensor or transducer arranged at a downstream position of the flow tube, and wherein the first piezoelectric sensor or transducer and the second piezoelectric sensor or transducer are configured to generate the time-series corresponding to the flow of the at least one fluid through the fluid flow path of the ultrasonic flow sensor.

In some non-limiting embodiments or aspects, the at least one fluid includes a sequence of fluids, wherein the time-series is generated by the ultrasonic flow sensor corresponding to a sequence of flows of the sequence of fluids through the fluid flow path of the ultrasonic flow sensor, and wherein the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof includes: at least one of (a) one or more predicted start times associated with one or more starts of one or more flows of one or more fluids of the sequence of fluids through the fluid flow path of the ultrasonic flow sensor, (b) one or more predicted time periods associated with the one or more flows of the one or more fluids of the sequence of fluids through the fluid flow path of the ultrasonic flow sensor, (c) one or more predicted end times associated with one or more ends of the one or more flows of the one or more fluids of the sequence of fluids through the fluid flow path of the ultrasonic flow sensor, or any combination thereof.

In some non-limiting embodiments or aspects, the time-series is further generated by the ultrasonic flow sensor corresponding to at least one period of no fluid flow through the fluid flow path of the ultrasonic flow sensor, and wherein the at least one processor is further configured to: generate, using the at least one machine learning model, based on the time-series, at least one of (x) a predicted no flow start time associated with a start of the at least one period of no fluid flow through the fluid flow path of the ultrasonic flow sensor, (y) a predicted no flow time period associated with the at least one period of no fluid flow through the fluid flow path of the ultrasonic flow sensor, (z) a predicted no flow end time associated with an end of the at least one period of no fluid flow through the fluid flow path of the ultrasonic flow sensor, or any combination thereof.

In some non-limiting embodiments or aspects, the at least one period of no fluid flow through the fluid flow path of the ultrasonic sensor occurs before the flow of the fluid of the sequence of fluids through the fluid flow path of the ultrasonic sensor.

In some non-limiting embodiments or aspects, the at least one period of no fluid flow through the fluid flow path of the ultrasonic sensor occurs after a flow of a fluid of the sequence of fluids through the fluid flow path of the ultrasonic sensor and before a flow of a next fluid of the sequence of fluids through the fluid flow path of the ultrasonic flow sensor.

In some non-limiting embodiments or aspects, the sequence of fluids includes at least one saline bolus and at least one medication bolus.

According to some non-limiting embodiments or aspects, provided is a method, including: receiving, with at least one processor, a time-series generated by an ultrasonic flow sensor corresponding to a flow of at least one fluid through a fluid flow path of the ultrasonic flow sensor; generating, with at least one processor, using at least one machine learning model, based on the time-series, at least one of (i) a predicted start time associated with a start of the flow of the at least one fluid through the fluid flow path of the ultrasonic flow sensor, (ii) a predicted time period associated with the flow of the at least one fluid through the fluid flow path of the ultrasonic flow sensor, (iii) a predicted end time associated with an end of the flow of the at least one fluid through the fluid flow path of the ultrasonic flow sensor, or any combination thereof; and providing, with at least one processor, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the end predicted time associated with the end of the flow of the at least one fluid through the fluid flow path of the ultrasonic flow sensor, or any combination thereof.

In some non-limiting embodiments or aspects, the time-series includes at least one of a plurality of amplitudes at a plurality of time points, a plurality of phases at the plurality of time points, or any combination thereof, and wherein the method further includes: generating, with the at least one processor, using at least one feature extraction algorithm, based on the time-series including the plurality of amplitudes at the plurality of time points, the plurality of phases at the plurality of time points, or any combination thereof, a feature representation associated with the time-series, and wherein generating, with the at least one processor, using the at least one machine learning model, based on the time-series, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, includes: providing, with the at least one processor, as input to the at least one machine learning model, the feature representation associated with the time series; and receiving, with the at least one processor, as output from the at least one machine learning model, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof.

In some non-limiting embodiments or aspects, the method further includes: identifying, with the at least one processor, based on the time series generated by the ultrasonic flow sensor corresponding to the flow of the at least one fluid through the fluid flow path of the ultrasonic flow sensor, in a database including a plurality of stored feature representations associated with a plurality of medication types, at least one stored feature representation of the plurality of stored feature representations, wherein the at least one stored feature representation is associated with at least one medication type; and providing, with the at least one processor, an indication of the at least one medication type.

In some non-limiting embodiments or aspects, the method further includes: obtaining, with the at least one processor, at least one expected medication type; determining, with the at least one processor, whether the at least one expected medication type corresponds to the at least one medication type associated with the identified at least one stored feature representation; and providing, with the at least one processor, an indication of whether the at least one expected medication type corresponds to the at least one medication type associated with the identified at least one stored feature representation.

In some non-limiting embodiments or aspects, generating, with the at least one processor, using the at least one machine learning model, based on the time-series, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, includes: generating, with the at least one processor, using the at least one machine learning model, based on the time-series and at least one of the at least one expected medication type, the at least one medication type, or any combination thereof, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof.

In some non-limiting embodiments or aspects, the method further includes: determining, with the at least one processor, based on the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, at least one of an amount of the at least one fluid that flowed through the fluid flow path of the ultrasonic flow sensor, a rate at which the at least one fluid flowed or is flowing through the fluid flow path of the ultrasonic flow sensor, or any combination thereof; and providing, with the at least one processor, the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof.

In some non-limiting embodiments or aspects, the method further includes: automatically storing, with the at least one processor, in a database, in association with a record of a patient, the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof.

In some non-limiting embodiments or aspects, the method further includes: determining, with the at least one processor, whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates at least one of a threshold amount associated with at least one fluid, a threshold rate associated with the at least one fluid, or any combination thereof; and providing, with the at least one processor, an indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof.

In some non-limiting embodiments or aspects, providing, with the at least one processor, the indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof, includes: controlling a user interface associated with the ultrasonic flow sensor to output at least one of an audio output, a visual output, or any combination thereof associated with whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof.

In some non-limiting embodiments or aspects, the method further includes: generating, with the ultrasonic flow sensor, the time-series corresponding to the flow of the at least one fluid through the fluid flow path of the ultrasonic flow sensor.

In some non-limiting embodiments or aspects, the at least one fluid includes a sequence of fluids, and wherein the method further includes: generating, with the ultrasonic flow sensor, the time-series corresponding to a sequence of flows of the sequence of fluids through the fluid flow path of the ultrasonic flow sensor, wherein the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof includes: at least one of (a) one or more predicted start times associated with one or more starts of one or more flows of one or more fluids of the sequence of fluids through the fluid flow path of the ultrasonic flow sensor, (b) one or more predicted time periods associated with the one or more flows of the one or more fluids of the sequence of fluids through the fluid flow path of the ultrasonic flow sensor, (c) one or more predicted end times associated with one or more ends of the one or more flows of the one or more fluids of the sequence of fluids through the fluid flow path of the ultrasonic flow sensor, or any combination thereof.

In some non-limiting embodiments or aspects, the method further includes: generating, with the ultrasonic flow senor, the time-series corresponding to at least one period of no fluid flow through the fluid flow path of the ultrasonic flow sensor; and generating, with the at least one processor, using the at least one machine learning model, based on the time-series, at least one of (x) a predicted no flow start time associated with a start of the at least one period of no fluid flow through the fluid flow path of the ultrasonic flow sensor, (y) a predicted no flow time period associated with the at least one period of no fluid flow through the fluid flow path of the ultrasonic flow sensor, (z) a predicted no flow end time associated with an end of the at least one period of no fluid flow through the fluid flow path of the ultrasonic flow sensor, or any combination thereof.

In some non-limiting embodiments or aspects, the at least one period of no fluid flow through the fluid flow path of the ultrasonic sensor occurs before the flow of the fluid of the sequence of fluids through the fluid flow path of the ultrasonic sensor.

In some non-limiting embodiments or aspects, the at least one period of no fluid flow through the fluid flow path of the ultrasonic sensor occurs after a flow of a fluid of the sequence of fluids through the fluid flow path of the ultrasonic sensor and before a flow of a next fluid of the sequence of fluids through the fluid flow path of the ultrasonic flow sensor.

In some non-limiting embodiments or aspects, the sequence of fluids includes at least one saline bolus and at least one medication bolus.

According to some non-limiting embodiments or aspects, provided is a computer program product including at least one non-transitory computer-readable medium including program instructions that, when executed by at least one processor, cause the at least one processor to: receive a time-series generated by an ultrasonic flow sensor corresponding to a flow of at least one fluid through a fluid flow path of the ultrasonic flow sensor; generate, using at least one machine learning model, based on the time-series, at least one of (i) a predicted start time associated with a start of the flow of the at least one fluid through the fluid flow path of the ultrasonic flow sensor, (ii) a predicted time period associated with the flow of the at least one fluid through the fluid flow path of the ultrasonic flow sensor, (iii) a predicted end time associated with an end of the flow of the at least one fluid through the fluid flow path of the ultrasonic flow sensor, or any combination thereof; and provide the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the end predicted time associated with the end of the flow of the at least one fluid through the fluid flow path of the ultrasonic flow sensor, or any combination thereof.

In some non-limiting embodiments or aspects, the time-series includes at least one of a plurality of amplitudes at a plurality of time points, a plurality of phases at the plurality of time points, or any combination thereof, and wherein the program instructions, when executed by the at least one processor, further cause the at least one processor to: generate, using at least one feature extraction algorithm, based on the time-series including the plurality of amplitudes at the plurality of time points, the plurality of phases at the plurality of time points, or any combination thereof, a feature representation associated with the time series, and wherein the program instructions, when executed by the at least one processor, cause the at least one processor to generate, using the at least one machine learning model, based on the time-series, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, by: providing, as input to the at least one machine learning model, the feature representation associated with the time series; and receiving, as output from the at least one machine learning model, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof.

In some non-limiting embodiments or aspects, the program instructions, when executed by the at least one processor, further cause the at least one processor to: identify, based on the time series generated by the ultrasonic flow sensor corresponding to the flow of the at least one fluid through the fluid flow path of the ultrasonic flow sensor, in a database including a plurality of stored feature representations associated with a plurality of medication types, at least one stored feature representation of the plurality of stored feature representations, wherein the at least one stored feature representation is associated with at least one medication type; and provide an indication of the at least one medication type.

In some non-limiting embodiments or aspects, the program instructions, when executed by the at least one processor, further cause the at least one processor to: obtain at least one expected medication type; determine whether the at least one expected medication type corresponds to the at least one medication type associated with the identified at least one stored feature representation; and provide an indication of whether the at least one expected medication type corresponds to the at least one medication type associated with the identified at least one stored feature representation.

In some non-limiting embodiments or aspects, the program instructions, when executed by the at least one processor, further cause the at least one processor to: generate, using the at least one machine learning model, based on the time-series and at least one of the at least one expected medication type, the at least one medication type, or any combination thereof, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof.

In some non-limiting embodiments or aspects, the program instructions, when executed by the at least one processor, further cause the at least one processor to: determine, based on the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, at least one of an amount of the at least one fluid that flowed through the fluid flow path of the ultrasonic flow sensor, a rate at which the at least one fluid flowed or is flowing through the fluid flow path of the ultrasonic flow sensor, or any combination thereof; and provide the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof.

In some non-limiting embodiments or aspects, the program instructions, when executed by the at least one processor, further cause the at least one processor to: automatically store, in a database, in association with a record of a patient, the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof.

In some non-limiting embodiments or aspects, the program instructions, when executed by the at least one processor, further cause the at least one processor to: determine whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates at least one of a threshold amount associated with at least one fluid, a threshold rate associated with the at least one fluid, or any combination thereof; and provide an indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof.

In some non-limiting embodiments or aspects, the program instructions, when executed by the at least one processor, cause the at least one processor to provide the indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof, by controlling a user interface associated with the ultrasonic flow sensor to output at least one of an audio output, a visual output, or any combination thereof associated with whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof.

In some non-limiting embodiments or aspects, the program instructions, when executed by the at least one processor, further cause the at least one processor to: control the ultrasonic flow sensor configured to generate the time-series corresponding to the flow of the at least one fluid through the fluid flow path of the ultrasonic flow sensor.

In some non-limiting embodiments or aspects, the at least one fluid includes a sequence of fluids, wherein the time-series is generated by the ultrasonic flow sensor corresponding to a sequence of flows of the sequence of fluids through the fluid flow path of the ultrasonic flow sensor, and wherein the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof includes: at least one of (a) one or more predicted start times associated with one or more starts of one or more flows of one or more fluids of the sequence of fluids through the fluid flow path of the ultrasonic flow sensor, (b) one or more predicted time periods associated with the one or more flows of the one or more fluids of the sequence of fluids through the fluid flow path of the ultrasonic flow sensor, (c) one or more predicted end times associated with one or more ends of the one or more flows of the one or more fluids of the sequence of fluids through the fluid flow path of the ultrasonic flow sensor, or any combination thereof.

In some non-limiting embodiments or aspects, the time-series is further generated by the ultrasonic flow sensor corresponding to at least one period of no fluid flow through the fluid flow path of the ultrasonic flow sensor, and wherein the program instructions, when executed by the at least one processor, further cause the at least one processor to: generate, using the at least one machine learning model, based on the time-series, at least one of (x) a predicted no flow start time associated with a start of the at least one period of no fluid flow through the fluid flow path of the ultrasonic flow sensor, (y) a predicted no flow time period associated with the at least one period of no fluid flow through the fluid flow path of the ultrasonic flow sensor, (z) a predicted no flow end time associated with an end of the at least one period of no fluid flow through the fluid flow path of the ultrasonic flow sensor, or any combination thereof.

In some non-limiting embodiments or aspects, the at least one period of no fluid flow through the fluid flow path of the ultrasonic sensor occurs before the flow of the fluid of the sequence of fluids through the fluid flow path of the ultrasonic sensor.

In some non-limiting embodiments or aspects, the at least one period of no fluid flow through the fluid flow path of the ultrasonic sensor occurs after a flow of a fluid of the sequence of fluids through the fluid flow path of the ultrasonic sensor and before a flow of a next fluid of the sequence of fluids through the fluid flow path of the ultrasonic flow sensor.

In some non-limiting embodiments or aspects, the sequence of fluids includes at least one saline bolus and at least one medication bolus.

According to some non-limiting embodiments or aspects, provided is a system, including: at least one processor configured to: receive a time-series generated by an ultrasonic flow sensor corresponding to a flow of at least one fluid through a fluid flow path of the ultrasonic flow sensor; identify, based on the time series generated by the ultrasonic flow sensor corresponding to the flow of the at least one fluid through the fluid flow path of the ultrasonic flow sensor, in a database including a plurality of stored feature representations associated with a plurality of medication types, at least one stored feature representation of the plurality of stored feature representations, wherein the at least one stored feature representation is associated with at least one medication type; and provide an indication of the at least one medication type.

In some non-limiting embodiments or aspects, the time-series includes at least one of a plurality of amplitudes at a plurality of time points, a plurality of phases at the plurality of time points, or any combination thereof, and wherein the at least one processor is further configured to: generate, using at least one feature extraction algorithm, based on the time-series including the plurality of amplitudes at the plurality of time points, the plurality of phases at the plurality of time points, or any combination thereof, a feature representation associated with the time-series, and wherein the at least one processor is configured to identify, based on the time series generated by the ultrasonic flow sensor corresponding to the flow of the at least one fluid through the fluid flow path of the ultrasonic flow sensor, in the database including the plurality of stored feature representations associated with the plurality of medication types, the at least one stored feature representation of the plurality of stored feature representations, by: identifying, in the database including the plurality of stored feature representations associated with the plurality of medication types, the at least one stored feature representation of the plurality of stored feature representations that corresponds to the feature representation associated with the time-series.

In some non-limiting embodiments or aspects, the at least one processor is configured to use a closed form expression to identify, in the database including the plurality of stored feature representations associated with the plurality of medication types, the at least one stored feature representation of the plurality of stored feature representations that corresponds to the feature representation associated with the time-series.

In some non-limiting embodiments or aspects, the at least one processor is configured to use one or more machine learning models to identify, in the database including the plurality of stored feature representations associated with the plurality of medication types, the at least one stored feature representation of the plurality of stored feature representations that corresponds to the feature representation associated with the time-series.

In some non-limiting embodiments or aspects, the at least one processor is further configured to: obtain at least one expected medication type; determine whether the at least one expected medication type corresponds to the at least one medication type associated with the identified at least one stored feature representation; and provide an indication of whether the at least one expected medication type corresponds to the at least one medication type associated with the identified at least one stored feature representation.

In some non-limiting embodiments or aspects, the system further includes: the ultrasonic flow sensor, wherein the ultrasonic flow sensor includes a flow tube that defines the fluid flow path of the ultrasonic flow sensor, a first piezoelectric sensor or transducer arranged at an upstream position of the flow tube, a second piezoelectric sensor or transducer arranged at a downstream position of the flow tube; and wherein the first piezoelectric sensor or transducer and the second piezoelectric sensor or transducer are configured to generate the time-series corresponding to the flow of the at least one fluid through the fluid flow path of the ultrasonic flow sensor.

In some non-limiting embodiments or aspects, the system further includes: at least one sensor configured to read medication information from a tag or label on a syringe, and wherein the medication information includes the at least one expected medication type.

In some non-limiting embodiments or aspects, the at least one processor is further configured to: generate, using at least one machine learning model, based on the time-series, at least one of (i) a predicted start time associated with a start of the flow of the at least one fluid through the fluid flow path of the ultrasonic flow sensor, (ii) a predicted time period associated with the flow of the at least one fluid through the fluid flow path of the ultrasonic flow sensor, (iii) a predicted end time associated with an end of the flow of the at least one fluid through the fluid flow path of the ultrasonic flow sensor, or any combination thereof; and provide the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the end predicted time associated with the end of the flow of the at least one fluid through the fluid flow path of the ultrasonic flow sensor, or any combination thereof.

According to some non-limiting embodiments or aspects, provided is a method, including: receiving, with at least one processor, a time-series generated by an ultrasonic flow sensor corresponding to a flow of at least one fluid through a fluid flow path of the ultrasonic flow sensor; identifying, with the at least one processor, based on the time series generated by the ultrasonic flow sensor corresponding to the flow of the at least one fluid through the fluid flow path of the ultrasonic flow sensor, in a database including a plurality of stored feature representations associated with a plurality of medication types, at least one stored feature representation of the plurality of stored feature representations, wherein the at least one stored feature representation is associated with at least one medication type; and providing, with the at least one processor, an indication of the at least one medication type.

In some non-limiting embodiments or aspects, the time-series includes at least one of a plurality of amplitudes at a plurality of time points, a plurality of phases at the plurality of time points, or any combination thereof, and wherein the method further includes: generating, with the at least one processor, using at least one feature extraction algorithm, based on the time-series including the plurality of amplitudes at the plurality of time points, the plurality of phases at the plurality of time points, or any combination thereof, a feature representation associated with the time-series, and wherein identifying, with the at least one processor, based on the time series generated by the ultrasonic flow sensor corresponding to the flow of the at least one fluid through the fluid flow path of the ultrasonic flow sensor, in the database including the plurality of stored feature representations associated with the plurality of medication types, the at least one stored feature representation of the plurality of stored feature representations, includes: identifying, with the at least one processor, in the database including the plurality of stored feature representations associated with the plurality of medication types, the at least one stored feature representation of the plurality of stored feature representations that corresponds to the feature representation associated with the time-series.

In some non-limiting embodiments or aspects, identifying, with the at least one processor, in the database including the plurality of stored feature representations associated with the plurality of medication types, the at least one stored feature representation of the plurality of stored feature representations that corresponds to the feature representation associated with the time-series, uses a closed form expression to identify the at least one stored feature representation of the plurality of stored feature representations that corresponds to the feature representation associated with the time-series.

In some non-limiting embodiments or aspects, the at least one processor is configured to use one or more machine learning models to identifying, with the at least one processor, in the database including the plurality of stored feature representations associated with the plurality of medication types, the at least one stored feature representation of the plurality of stored feature representations that corresponds to the feature representation associated with the time-series, uses one or more machine learning models to identify the at least one stored feature representation of the plurality of stored feature representations that corresponds to the feature representation associated with the time-series.

In some non-limiting embodiments or aspects, the method further includes: obtaining, with the at least one processor, at least one expected medication type; determining, with the at least one processor, whether the at least one expected medication type corresponds to the at least one medication type associated with the identified at least one stored feature representation; and providing, with the at least one processor, an indication of whether the at least one expected medication type corresponds to the at least one medication type associated with the identified at least one stored feature representation.

In some non-limiting embodiments or aspects, the method further includes: generating, with the ultrasonic flow sensor, the time-series corresponding to the flow of the at least one fluid through the fluid flow path of the ultrasonic flow sensor, wherein the ultrasonic flow sensor includes a flow tube that defines the fluid flow path of the ultrasonic flow sensor, a first piezoelectric sensor or transducer arranged at an upstream position of the flow tube, and a second piezoelectric sensor or transducer arranged at a downstream position of the flow tube, and wherein the first piezoelectric sensor or transducer and the second piezoelectric sensor or transducer of the ultrasonic flow sensor generate the time-series corresponding to the flow of the at least one fluid through the fluid flow path of the ultrasonic flow sensor.

In some non-limiting embodiments or aspects, the method further includes: reading, with at least one sensor, medication information from a tag or label on a syringe, wherein the medication information includes the at least one expected medication type.

In some non-limiting embodiments or aspects, the method further includes: generating, with the at least one processor, using at least one machine learning model, based on the time-series, at least one of (i) a predicted start time associated with a start of the flow of the at least one fluid through the fluid flow path of the ultrasonic flow sensor, (ii) a predicted time period associated with the flow of the at least one fluid through the fluid flow path of the ultrasonic flow sensor, (iii) a predicted end time associated with an end of the flow of the at least one fluid through the fluid flow path of the ultrasonic flow sensor, or any combination thereof; and providing, with the at least one processor, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the end predicted time associated with the end of the flow of the at least one fluid through the fluid flow path of the ultrasonic flow sensor, or any combination thereof.

According to some non-limiting embodiments or aspects, provided is a computer program product including at least one non-transitory computer-readable medium including program instructions that, when executed by at least one processor, cause the at least one processor to: receive a time-series generated by an ultrasonic flow sensor corresponding to a flow of at least one fluid through a fluid flow path of the ultrasonic flow sensor; identify, based on the time series generated by the ultrasonic flow sensor corresponding to the flow of the at least one fluid through the fluid flow path of the ultrasonic flow sensor, in a database including a plurality of stored feature representations associated with a plurality of medication types, at least one stored feature representation of the plurality of stored feature representations, wherein the at least one stored feature representation is associated with at least one medication type; and provide an indication of the at least one medication type.

In some non-limiting embodiments or aspects, the time-series includes at least one of a plurality of amplitudes at a plurality of time points, a plurality of phases at the plurality of time points, or any combination thereof, and wherein the program instructions, when executed by the at least one processor, further cause the at least one processor to: generate, using at least one feature extraction algorithm, based on the time-series including the plurality of amplitudes at the plurality of time points, the plurality of phases at the plurality of time points, or any combination thereof, a feature representation associated with the time-series, and wherein the at least one processor is configured to identify, based on the time series generated by the ultrasonic flow sensor corresponding to the flow of the at least one fluid through the fluid flow path of the ultrasonic flow sensor, in the database including the plurality of stored feature representations associated with the plurality of medication types, the at least one stored feature representation of the plurality of stored feature representations, by: identifying, in the database including the plurality of stored feature representations associated with the plurality of medication types, the at least one stored feature representation of the plurality of stored feature representations that corresponds to the feature representation associated with the time-series.

In some non-limiting embodiments or aspects, the program instructions, when executed by the at least one processor, cause the at least one processor to use a closed form expression to identify, in the database including the plurality of stored feature representations associated with the plurality of medication types, the at least one stored feature representation of the plurality of stored feature representations that corresponds to the feature representation associated with the time-series.

In some non-limiting embodiments or aspects, the program instructions, when executed by the at least one processor, cause the at least one processor to use one or more machine learning models to identify, in the database including the plurality of stored feature representations associated with the plurality of medication types, the at least one stored feature representation of the plurality of stored feature representations that corresponds to the feature representation associated with the time-series.

In some non-limiting embodiments or aspects, the program instructions, when executed by the at least one processor, further cause the at least one processor to: obtain at least one expected medication type; determine whether the at least one expected medication type corresponds to the at least one medication type associated with the identified at least one stored feature representation; and provide an indication of whether the at least one expected medication type corresponds to the at least one medication type associated with the identified at least one stored feature representation.

In some non-limiting embodiments or aspects, the program instructions, when executed by the at least one processor, further cause the at least one processor to: control the ultrasonic flow sensor to generate the time-series corresponding to the flow of the at least one fluid through the fluid flow path of the ultrasonic flow sensor, wherein the ultrasonic flow sensor includes a flow tube that defines the fluid flow path of the ultrasonic flow sensor, a first piezoelectric sensor or transducer arranged at an upstream position of the flow tube, and a second piezoelectric sensor or transducer arranged at a downstream position of the flow tube, and wherein the first piezoelectric sensor or transducer and the second piezoelectric sensor or transducer of the ultrasonic flow sensor generate the time-series corresponding to the flow of the at least one fluid through the fluid flow path of the ultrasonic flow sensor.

In some non-limiting embodiments or aspects, the program instructions, when executed by the at least one processor, further cause the at least one processor to: control at least one sensor to read medication information from a tag or label on a syringe, and wherein the medication information includes the at least one expected medication type.

In some non-limiting embodiments or aspects, the program instructions, when executed by the at least one processor, further cause the at least one processor to: generate, using at least one machine learning model, based on the time-series, at least one of (i) a predicted start time associated with a start of the flow of the at least one fluid through the fluid flow path of the ultrasonic flow sensor, (ii) a predicted time period associated with the flow of the at least one fluid through the fluid flow path of the ultrasonic flow sensor, (iii) a predicted end time associated with an end of the flow of the at least one fluid through the fluid flow path of the ultrasonic flow sensor, or any combination thereof; and provide the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the end predicted time associated with the end of the flow of the at least one fluid through the fluid flow path of the ultrasonic flow sensor, or any combination thereof.

Further non-limiting embodiments or aspects are set forth in the following numbered clauses:

Clause 1: A system, comprising: at least one processor configured to: receive a time-series generated by an ultrasonic flow sensor corresponding to a flow of at least one fluid through a fluid flow path of the ultrasonic flow sensor; generate, using at least one machine learning model, based on the time-series, at least one of (i) a predicted start time associated with a start of the flow of the at least one fluid through the fluid flow path of the ultrasonic flow sensor, (ii) a predicted time period associated with the flow of the at least one fluid through the fluid flow path of the ultrasonic flow sensor, (iii) a predicted end time associated with an end of the flow of the at least one fluid through the fluid flow path of the ultrasonic flow sensor, or any combination thereof; and provide the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the end predicted time associated with the end of the flow of the at least one fluid through the fluid flow path of the ultrasonic flow sensor, or any combination thereof.

Clause 2: The system of clause 1, wherein the time-series includes at least one of a plurality of amplitudes at a plurality of time points, a plurality of phases at the plurality of time points, or any combination thereof, and wherein the at least one processor is further configured to: generate, using at least one feature extraction algorithm, based on the time-series including the plurality of amplitudes at the plurality of time points, the plurality of phases at the plurality of time points, or any combination thereof, a feature representation associated with the time-series, and wherein the at least one processor is configured to generate, using the at least one machine learning model, based on the time-series, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, by: providing, as input to the at least one machine learning model, the feature representation associated with the time-series; and receiving, as output from the at least one machine learning model, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof.

Clause 3: The system of clause 1 or clause 2, wherein the at least one processor is further configured to: identify, based on the time series generated by the ultrasonic flow sensor corresponding to the flow of the at least one fluid through the fluid flow path of the ultrasonic flow sensor, in a database including a plurality of stored feature representations associated with a plurality of medication types, at least one stored feature representation of the plurality of stored feature representations, wherein the at least one stored feature representation is associated with at least one medication type; and provide an indication of the at least one medication type.

Clause 4. The system of any of clauses 1-3, wherein the at least one processor is further configured to: obtain at least one expected medication type; determine whether the at least one expected medication type corresponds to the at least one medication type associated with the identified at least one stored feature representation; and provide an indication of whether the at least one expected medication type corresponds to the at least one medication type associated with the identified at least one stored feature representation.

Clause 5: The system of any of clauses 1-4, wherein the at least one processor is configured to generate, using the at least one machine learning model, based on the time-series and at least one of the at least one expected medication type, the at least one medication type, or any combination thereof, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof.

Clause 6: The system of any of clauses 1-5, wherein the at least one processor is further configured to: determine, based on the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, at least one of an amount of the at least one fluid that flowed through the fluid flow path of the ultrasonic flow sensor, a rate at which the at least one fluid flowed or is flowing through the fluid flow path of the ultrasonic flow sensor, or any combination thereof; and provide the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof.

Clause 7: The system of any of clauses 1-6, wherein the at least one processor is further configured to: automatically store, in a database, in association with a record of a patient, the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof.

Clause 8: The system of any of clauses 1-7, wherein the at least one processor is further configured to: determine whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates at least one of a threshold amount associated with at least one fluid, a threshold rate associated with the at least one fluid, or any combination thereof; and provide an indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof.

Clause 9: The system of any of clauses 1-8, wherein the at least one processor is configured to provide the indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof, by controlling a user interface associated with the ultrasonic flow sensor to output at least one of an audio output, a visual output, or any combination thereof associated with whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof.

Clause 10: The system of any of clauses 1-9, further comprising: the ultrasonic flow sensor, wherein the ultrasonic flow sensor includes a flow tube that defines the fluid flow path of the ultrasonic flow sensor, a first piezoelectric sensor or transducer arranged at an upstream position of the flow tube, and a second piezoelectric sensor or transducer arranged at a downstream position of the flow tube, and wherein the first piezoelectric sensor or transducer and the second piezoelectric sensor or transducer are configured to generate the time-series corresponding to the flow of the at least one fluid through the fluid flow path of the ultrasonic flow sensor.

Clause 11: The system of any of clauses 1-10, wherein the at least one fluid includes a sequence of fluids, wherein the time-series is generated by the ultrasonic flow sensor corresponding to a sequence of flows of the sequence of fluids through the fluid flow path of the ultrasonic flow sensor, and wherein the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof includes: at least one of (a) one or more predicted start times associated with one or more starts of one or more flows of one or more fluids of the sequence of fluids through the fluid flow path of the ultrasonic flow sensor, (b) one or more predicted time periods associated with the one or more flows of the one or more fluids of the sequence of fluids through the fluid flow path of the ultrasonic flow sensor, (c) one or more predicted end times associated with one or more ends of the one or more flows of the one or more fluids of the sequence of fluids through the fluid flow path of the ultrasonic flow sensor, or any combination thereof.

Clause 12: The system of any of clauses 1-11, wherein the time-series is further generated by the ultrasonic flow sensor corresponding to at least one period of no fluid flow through the fluid flow path of the ultrasonic flow sensor, and wherein the at least one processor is further configured to: generate, using the at least one machine learning model, based on the time-series, at least one of (x) a predicted no flow start time associated with a start of the at least one period of no fluid flow through the fluid flow path of the ultrasonic flow sensor, (y) a predicted no flow time period associated with the at least one period of no fluid flow through the fluid flow path of the ultrasonic flow sensor, (z) a predicted no flow end time associated with an end of the at least one period of no fluid flow through the fluid flow path of the ultrasonic flow sensor, or any combination thereof.

Clause 13: The system of any of clauses 1-12, wherein the at least one period of no fluid flow through the fluid flow path of the ultrasonic sensor occurs before the flow of the fluid of the sequence of fluids through the fluid flow path of the ultrasonic sensor.

Clause 14: The system of any of clauses 1-13, wherein the at least one period of no fluid flow through the fluid flow path of the ultrasonic sensor occurs after a flow of a fluid of the sequence of fluids through the fluid flow path of the ultrasonic sensor and before a flow of a next fluid of the sequence of fluids through the fluid flow path of the ultrasonic flow sensor.

Clause 15: The system of any of clauses 1-14, wherein the sequence of fluids includes at least one saline bolus and at least one medication bolus.

Clause 16: A method, comprising: receiving, with at least one processor, a time-series generated by an ultrasonic flow sensor corresponding to a flow of at least one fluid through a fluid flow path of the ultrasonic flow sensor; generating, with at least one processor, using at least one machine learning model, based on the time-series, at least one of (i) a predicted start time associated with a start of the flow of the at least one fluid through the fluid flow path of the ultrasonic flow sensor, (ii) a predicted time period associated with the flow of the at least one fluid through the fluid flow path of the ultrasonic flow sensor, (iii) a predicted end time associated with an end of the flow of the at least one fluid through the fluid flow path of the ultrasonic flow sensor, or any combination thereof; and providing, with at least one processor, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the end predicted time associated with the end of the flow of the at least one fluid through the fluid flow path of the ultrasonic flow sensor, or any combination thereof.

Clause 17: The method of clause 16, wherein the time-series includes at least one of a plurality of amplitudes at a plurality of time points, a plurality of phases at the plurality of time points, or any combination thereof, and wherein the method further comprises: generating, with the at least one processor, using at least one feature extraction algorithm, based on the time-series including the plurality of amplitudes at the plurality of time points, the plurality of phases at the plurality of time points, or any combination thereof, a feature representation associated with the time-series, and wherein generating, with the at least one processor, using the at least one machine learning model, based on the time-series, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, includes: providing, with the at least one processor, as input to the at least one machine learning model, the feature representation associated with the time series; and receiving, with the at least one processor, as output from the at least one machine learning model, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof.

Clause 18: The method of clause 16 or clause 17, further comprising: identifying, with the at least one processor, based on the time series generated by the ultrasonic flow sensor corresponding to the flow of the at least one fluid through the fluid flow path of the ultrasonic flow sensor, in a database including a plurality of stored feature representations associated with a plurality of medication types, at least one stored feature representation of the plurality of stored feature representations, wherein the at least one stored feature representation is associated with at least one medication type; and providing, with the at least one processor, an indication of the at least one medication type.

Clause 19: The method of any of clauses 16-18, further comprising: obtaining, with the at least one processor, at least one expected medication type; determining, with the at least one processor, whether the at least one expected medication type corresponds to the at least one medication type associated with the identified at least one stored feature representation; and providing, with the at least one processor, an indication of whether the at least one expected medication type corresponds to the at least one medication type associated with the identified at least one stored feature representation.

Clause 20: The method of any of clauses 16-19, wherein generating, with the at least one processor, using the at least one machine learning model, based on the time-series, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, includes: generating, with the at least one processor, using the at least one machine learning model, based on the time-series and at least one of the at least one expected medication type, the at least one medication type, or any combination thereof, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof.

Clause 21: The method of any of clauses 16-20, further comprising: determining, with the at least one processor, based on the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, at least one of an amount of the at least one fluid that flowed through the fluid flow path of the ultrasonic flow sensor, a rate at which the at least one fluid flowed or is flowing through the fluid flow path of the ultrasonic flow sensor, or any combination thereof; and providing, with the at least one processor, the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof.

Clause 22: The method of any of clauses 16-21, further comprising: automatically storing, with the at least one processor, in a database, in association with a record of a patient, the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof.

Clause 23: The method of any of clauses 16-22, further comprising: determining, with the at least one processor, whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates at least one of a threshold amount associated with at least one fluid, a threshold rate associated with the at least one fluid, or any combination thereof; and providing, with the at least one processor, an indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof.

Clause 24: The method of any of clauses 16-23, wherein providing, with the at least one processor, the indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof, includes: controlling a user interface associated with the ultrasonic flow sensor to output at least one of an audio output, a visual output, or any combination thereof associated with whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof.

Clause 25: The method of any of clauses 16-24, further comprising: generating, with the ultrasonic flow sensor, the time-series corresponding to the flow of the at least one fluid through the fluid flow path of the ultrasonic flow sensor.

Clause 26: The method of any of clauses 16-25, wherein the at least one fluid includes a sequence of fluids, and wherein the method further comprises: generating, with the ultrasonic flow sensor, the time-series corresponding to a sequence of flows of the sequence of fluids through the fluid flow path of the ultrasonic flow sensor, wherein the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof includes: at least one of (a) one or more predicted start times associated with one or more starts of one or more flows of one or more fluids of the sequence of fluids through the fluid flow path of the ultrasonic flow sensor, (b) one or more predicted time periods associated with the one or more flows of the one or more fluids of the sequence of fluids through the fluid flow path of the ultrasonic flow sensor, (c) one or more predicted end times associated with one or more ends of the one or more flows of the one or more fluids of the sequence of fluids through the fluid flow path of the ultrasonic flow sensor, or any combination thereof.

Clause 27: The method of any of clauses 16-26, further comprising: generating, with the ultrasonic flow senor, the time-series corresponding to at least one period of no fluid flow through the fluid flow path of the ultrasonic flow sensor; and generating, with the at least one processor, using the at least one machine learning model, based on the time-series, at least one of (x) a predicted no flow start time associated with a start of the at least one period of no fluid flow through the fluid flow path of the ultrasonic flow sensor, (y) a predicted no flow time period associated with the at least one period of no fluid flow through the fluid flow path of the ultrasonic flow sensor, (z) a predicted no flow end time associated with an end of the at least one period of no fluid flow through the fluid flow path of the ultrasonic flow sensor, or any combination thereof.

Clause 28: The method of any of clauses 16-27, wherein the at least one period of no fluid flow through the fluid flow path of the ultrasonic sensor occurs before the flow of the fluid of the sequence of fluids through the fluid flow path of the ultrasonic sensor.

Clause 29: The method of any of clauses 16-28, wherein the at least one period of no fluid flow through the fluid flow path of the ultrasonic sensor occurs after a flow of a fluid of the sequence of fluids through the fluid flow path of the ultrasonic sensor and before a flow of a next fluid of the sequence of fluids through the fluid flow path of the ultrasonic flow sensor.

Clause 30: The method of any of clauses 16-29, wherein the sequence of fluids includes at least one saline bolus and at least one medication bolus.

Clause 31: A computer program product comprising at least one non-transitory computer-readable medium including program instructions that, when executed by at least one processor, cause the at least one processor to: receive a time-series generated by an ultrasonic flow sensor corresponding to a flow of at least one fluid through a fluid flow path of the ultrasonic flow sensor; generate, using at least one machine learning model, based on the time-series, at least one of (i) a predicted start time associated with a start of the flow of the at least one fluid through the fluid flow path of the ultrasonic flow sensor, (ii) a predicted time period associated with the flow of the at least one fluid through the fluid flow path of the ultrasonic flow sensor, (iii) a predicted end time associated with an end of the flow of the at least one fluid through the fluid flow path of the ultrasonic flow sensor, or any combination thereof; and provide the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the end predicted time associated with the end of the flow of the at least one fluid through the fluid flow path of the ultrasonic flow sensor, or any combination thereof.

Clause 32: The computer program product of clause 31, wherein the time-series includes at least one of a plurality of amplitudes at a plurality of time points, a plurality of phases at the plurality of time points, or any combination thereof, and wherein the program instructions, when executed by the at least one processor, further cause the at least one processor to: generate, using at least one feature extraction algorithm, based on the time-series including the plurality of amplitudes at the plurality of time points, the plurality of phases at the plurality of time points, or any combination thereof, a feature representation associated with the time series, and wherein the program instructions, when executed by the at least one processor, cause the at least one processor to generate, using the at least one machine learning model, based on the time-series, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, by: providing, as input to the at least one machine learning model, the feature representation associated with the time series; and receiving, as output from the at least one machine learning model, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof.

Clause 33: The computer program product of clause 31 or clause 32, wherein the program instructions, when executed by the at least one processor, further cause the at least one processor to: identify, based on the time series generated by the ultrasonic flow sensor corresponding to the flow of the at least one fluid through the fluid flow path of the ultrasonic flow sensor, in a database including a plurality of stored feature representations associated with a plurality of medication types, at least one stored feature representation of the plurality of stored feature representations, wherein the at least one stored feature representation is associated with at least one medication type; and provide an indication of the at least one medication type.

Clause 34: The computer program product of any of clauses 31-33, wherein the program instructions, when executed by the at least one processor, further cause the at least one processor to: obtain at least one expected medication type; determine whether the at least one expected medication type corresponds to the at least one medication type associated with the identified at least one stored feature representation; and provide an indication of whether the at least one expected medication type corresponds to the at least one medication type associated with the identified at least one stored feature representation.

Clause 35: The computer program product of any of clauses 31-34, wherein the program instructions, when executed by the at least one processor, further cause the at least one processor to: generate, using the at least one machine learning model, based on the time-series and at least one of the at least one expected medication type, the at least one medication type, or any combination thereof, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof.

Clause 36: The computer program product of any of clauses 31-35, wherein the program instructions, when executed by the at least one processor, further cause the at least one processor to: determine, based on the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, at least one of an amount of the at least one fluid that flowed through the fluid flow path of the ultrasonic flow sensor, a rate at which the at least one fluid flowed or is flowing through the fluid flow path of the ultrasonic flow sensor, or any combination thereof; and provide the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof.

Clause 37: The computer program product of any of clauses 31-36, wherein the program instructions, when executed by the at least one processor, further cause the at least one processor to: automatically store, in a database, in association with a record of a patient, the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof.

Clause 38: The computer program product of any of clauses 31-37, wherein the program instructions, when executed by the at least one processor, further cause the at least one processor to: determine whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates at least one of a threshold amount associated with at least one fluid, a threshold rate associated with the at least one fluid, or any combination thereof; and provide an indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof.

Clause 39: The computer program product of any of clauses 31-38, wherein the program instructions, when executed by the at least one processor, cause the at least one processor to provide the indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof, by controlling a user interface associated with the ultrasonic flow sensor to output at least one of an audio output, a visual output, or any combination thereof associated with whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof.

Clause 40: The computer program product of any of clauses 31-39, wherein the program instructions, when executed by the at least one processor, further cause the at least one processor to: control the ultrasonic flow sensor configured to generate the time-series corresponding to the flow of the at least one fluid through the fluid flow path of the ultrasonic flow sensor.

Clause 41: The computer program product of any of clauses 31-40, wherein the at least one fluid includes a sequence of fluids, wherein the time-series is generated by the ultrasonic flow sensor corresponding to a sequence of flows of the sequence of fluids through the fluid flow path of the ultrasonic flow sensor, and wherein the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof includes: at least one of (a) one or more predicted start times associated with one or more starts of one or more flows of one or more fluids of the sequence of fluids through the fluid flow path of the ultrasonic flow sensor, (b) one or more predicted time periods associated with the one or more flows of the one or more fluids of the sequence of fluids through the fluid flow path of the ultrasonic flow sensor, (c) one or more predicted end times associated with one or more ends of the one or more flows of the one or more fluids of the sequence of fluids through the fluid flow path of the ultrasonic flow sensor, or any combination thereof.

Clause 42: The computer program product of any of clauses 31-41, wherein the time-series is further generated by the ultrasonic flow sensor corresponding to at least one period of no fluid flow through the fluid flow path of the ultrasonic flow sensor, and wherein the program instructions, when executed by the at least one processor, further cause the at least one processor to: generate, using the at least one machine learning model, based on the time-series, at least one of (x) a predicted no flow start time associated with a start of the at least one period of no fluid flow through the fluid flow path of the ultrasonic flow sensor, (y) a predicted no flow time period associated with the at least one period of no fluid flow through the fluid flow path of the ultrasonic flow sensor, (z) a predicted no flow end time associated with an end of the at least one period of no fluid flow through the fluid flow path of the ultrasonic flow sensor, or any combination thereof.

Clause 43: The computer program product of any of clauses 31-42, wherein the at least one period of no fluid flow through the fluid flow path of the ultrasonic sensor occurs before the flow of the fluid of the sequence of fluids through the fluid flow path of the ultrasonic sensor.

Clause 44: The computer program product of any of clauses 31-43, wherein the at least one period of no fluid flow through the fluid flow path of the ultrasonic sensor occurs after a flow of a fluid of the sequence of fluids through the fluid flow path of the ultrasonic sensor and before a flow of a next fluid of the sequence of fluids through the fluid flow path of the ultrasonic flow sensor.

Clause 45: The computer program product of any of clauses 31-44, wherein the sequence of fluids includes at least one saline bolus and at least one medication bolus.

Clause 46: A system, comprising: at least one processor configured to: receive a time-series generated by an ultrasonic flow sensor corresponding to a flow of at least one fluid through a fluid flow path of the ultrasonic flow sensor; identify, based on the time series generated by the ultrasonic flow sensor corresponding to the flow of the at least one fluid through the fluid flow path of the ultrasonic flow sensor, in a database including a plurality of stored feature representations associated with a plurality of medication types, at least one stored feature representation of the plurality of stored feature representations, wherein the at least one stored feature representation is associated with at least one medication type; and provide an indication of the at least one medication type.

Clause 47: The system of clause 46, wherein the time-series includes at least one of a plurality of amplitudes at a plurality of time points, a plurality of phases at the plurality of time points, or any combination thereof, and wherein the at least one processor is further configured to: generate, using at least one feature extraction algorithm, based on the time-series including the plurality of amplitudes at the plurality of time points, the plurality of phases at the plurality of time points, or any combination thereof, a feature representation associated with the time-series, and wherein the at least one processor is configured to identify, based on the time series generated by the ultrasonic flow sensor corresponding to the flow of the at least one fluid through the fluid flow path of the ultrasonic flow sensor, in the database including the plurality of stored feature representations associated with the plurality of medication types, the at least one stored feature representation of the plurality of stored feature representations, by: identifying, in the database including the plurality of stored feature representations associated with the plurality of medication types, the at least one stored feature representation of the plurality of stored feature representations that corresponds to the feature representation associated with the time-series.

Clause 48: The system of clause 46 or clause 47, wherein the at least one processor is configured to use a closed form expression to identify, in the database including the plurality of stored feature representations associated with the plurality of medication types, the at least one stored feature representation of the plurality of stored feature representations that corresponds to the feature representation associated with the time-series.

Clause 49: The system of any of clauses 46-48, wherein the at least one processor is configured to use one or more machine learning models to identify, in the database including the plurality of stored feature representations associated with the plurality of medication types, the at least one stored feature representation of the plurality of stored feature representations that corresponds to the feature representation associated with the time-series.

Clause 50: The system of any of clauses 46-49, wherein the at least one processor is further configured to: obtain at least one expected medication type; determine whether the at least one expected medication type corresponds to the at least one medication type associated with the identified at least one stored feature representation; and provide an indication of whether the at least one expected medication type corresponds to the at least one medication type associated with the identified at least one stored feature representation.

Clause 51: The system of any of clauses 46-50, further comprising: the ultrasonic flow sensor, wherein the ultrasonic flow sensor includes a flow tube that defines the fluid flow path of the ultrasonic flow sensor, a first piezoelectric sensor or transducer arranged at an upstream position of the flow tube, a second piezoelectric sensor or transducer arranged at a downstream position of the flow tube; and wherein the first piezoelectric sensor or transducer and the second piezoelectric sensor or transducer are configured to generate the time-series corresponding to the flow of the at least one fluid through the fluid flow path of the ultrasonic flow sensor.

Clause 52: The system of any of clauses 46-51, further comprising: at least one sensor configured to read medication information from a tag or label on a syringe, and wherein the medication information includes the at least one expected medication type.

Clause 53: The system of any of clauses 46-52, wherein the at least one processor is further configured to: generate, using at least one machine learning model, based on the time-series, at least one of (i) a predicted start time associated with a start of the flow of the at least one fluid through the fluid flow path of the ultrasonic flow sensor, (ii) a predicted time period associated with the flow of the at least one fluid through the fluid flow path of the ultrasonic flow sensor, (iii) a predicted end time associated with an end of the flow of the at least one fluid through the fluid flow path of the ultrasonic flow sensor, or any combination thereof; and provide the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the end predicted time associated with the end of the flow of the at least one fluid through the fluid flow path of the ultrasonic flow sensor, or any combination thereof.

Clause 54: A method, comprising: receiving, with at least one processor, a time-series generated by an ultrasonic flow sensor corresponding to a flow of at least one fluid through a fluid flow path of the ultrasonic flow sensor; identifying, with the at least one processor, based on the time series generated by the ultrasonic flow sensor corresponding to the flow of the at least one fluid through the fluid flow path of the ultrasonic flow sensor, in a database including a plurality of stored feature representations associated with a plurality of medication types, at least one stored feature representation of the plurality of stored feature representations, wherein the at least one stored feature representation is associated with at least one medication type; and providing, with the at least one processor, an indication of the at least one medication type.

Clause 55: The method of clause 54, wherein the time-series includes at least one of a plurality of amplitudes at a plurality of time points, a plurality of phases at the plurality of time points, or any combination thereof, and wherein the method further comprises: generating, with the at least one processor, using at least one feature extraction algorithm, based on the time-series including the plurality of amplitudes at the plurality of time points, the plurality of phases at the plurality of time points, or any combination thereof, a feature representation associated with the time-series, and wherein identifying, with the at least one processor, based on the time series generated by the ultrasonic flow sensor corresponding to the flow of the at least one fluid through the fluid flow path of the ultrasonic flow sensor, in the database including the plurality of stored feature representations associated with the plurality of medication types, the at least one stored feature representation of the plurality of stored feature representations, includes: identifying, with the at least one processor, in the database including the plurality of stored feature representations associated with the plurality of medication types, the at least one stored feature representation of the plurality of stored feature representations that corresponds to the feature representation associated with the time-series.

Clause 56: The method of clause 54 or clause 55, wherein identifying, with the at least one processor, in the database including the plurality of stored feature representations associated with the plurality of medication types, the at least one stored feature representation of the plurality of stored feature representations that corresponds to the feature representation associated with the time-series, uses a closed form expression to identify the at least one stored feature representation of the plurality of stored feature representations that corresponds to the feature representation associated with the time-series.

Clause 57. The method of any of clauses 54-56, wherein the at least one processor is configured to use one or more machine learning models to identifying, with the at least one processor, in the database including the plurality of stored feature representations associated with the plurality of medication types, the at least one stored feature representation of the plurality of stored feature representations that corresponds to the feature representation associated with the time-series, uses one or more machine learning models to identify the at least one stored feature representation of the plurality of stored feature representations that corresponds to the feature representation associated with the time-series.

Clause 58: The method of any of clauses 54-57, further comprising: obtaining, with the at least one processor, at least one expected medication type; determining, with the at least one processor, whether the at least one expected medication type corresponds to the at least one medication type associated with the identified at least one stored feature representation; and providing, with the at least one processor, an indication of whether the at least one expected medication type corresponds to the at least one medication type associated with the identified at least one stored feature representation.

Clause 59: The method of any of clauses 54-58, further comprising: generating, with the ultrasonic flow sensor, the time-series corresponding to the flow of the at least one fluid through the fluid flow path of the ultrasonic flow sensor, wherein the ultrasonic flow sensor includes a flow tube that defines the fluid flow path of the ultrasonic flow sensor, a first piezoelectric sensor or transducer arranged at an upstream position of the flow tube, and a second piezoelectric sensor or transducer arranged at a downstream position of the flow tube, and wherein the first piezoelectric sensor or transducer and the second piezoelectric sensor or transducer of the ultrasonic flow sensor generate the time-series corresponding to the flow of the at least one fluid through the fluid flow path of the ultrasonic flow sensor.

Clause 60: The method of any of clauses 54-59, further comprising: reading, with at least one sensor, medication information from a tag or label on a syringe, wherein the medication information includes the at least one expected medication type.

Clause 61: The method of any of clauses 54-60, further comprising: generating, with the at least one processor, using at least one machine learning model, based on the time-series, at least one of (i) a predicted start time associated with a start of the flow of the at least one fluid through the fluid flow path of the ultrasonic flow sensor, (ii) a predicted time period associated with the flow of the at least one fluid through the fluid flow path of the ultrasonic flow sensor, (iii) a predicted end time associated with an end of the flow of the at least one fluid through the fluid flow path of the ultrasonic flow sensor, or any combination thereof; and providing, with the at least one processor, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the end predicted time associated with the end of the flow of the at least one fluid through the fluid flow path of the ultrasonic flow sensor, or any combination thereof.

Clause 62: A computer program product comprising at least one non-transitory computer-readable medium including program instructions that, when executed by at least one processor, cause the at least one processor to: receive a time-series generated by an ultrasonic flow sensor corresponding to a flow of at least one fluid through a fluid flow path of the ultrasonic flow sensor; identify, based on the time series generated by the ultrasonic flow sensor corresponding to the flow of the at least one fluid through the fluid flow path of the ultrasonic flow sensor, in a database including a plurality of stored feature representations associated with a plurality of medication types, at least one stored feature representation of the plurality of stored feature representations, wherein the at least one stored feature representation is associated with at least one medication type; and provide an indication of the at least one medication type.

Clause 63: The computer program product of clause 62, wherein the time-series includes at least one of a plurality of amplitudes at a plurality of time points, a plurality of phases at the plurality of time points, or any combination thereof, and wherein the program instructions, when executed by the at least one processor, further cause the at least one processor to: generate, using at least one feature extraction algorithm, based on the time-series including the plurality of amplitudes at the plurality of time points, the plurality of phases at the plurality of time points, or any combination thereof, a feature representation associated with the time-series, and wherein the at least one processor is configured to identify, based on the time series generated by the ultrasonic flow sensor corresponding to the flow of the at least one fluid through the fluid flow path of the ultrasonic flow sensor, in the database including the plurality of stored feature representations associated with the plurality of medication types, the at least one stored feature representation of the plurality of stored feature representations, by: identifying, in the database including the plurality of stored feature representations associated with the plurality of medication types, the at least one stored feature representation of the plurality of stored feature representations that corresponds to the feature representation associated with the time-series.

Clause 64: The computer program product of clause 62 or clause 63, wherein the program instructions, when executed by the at least one processor, cause the at least one processor to use a closed form expression to identify, in the database including the plurality of stored feature representations associated with the plurality of medication types, the at least one stored feature representation of the plurality of stored feature representations that corresponds to the feature representation associated with the time-series.

Clause 65: The computer program product of any of clauses 62-64, wherein the program instructions, when executed by the at least one processor, cause the at least one processor to use one or more machine learning models to identify, in the database including the plurality of stored feature representations associated with the plurality of medication types, the at least one stored feature representation of the plurality of stored feature representations that corresponds to the feature representation associated with the time-series.

Clause 66: The computer program product of any of clauses 62-65, wherein the program instructions, when executed by the at least one processor, further cause the at least one processor to: obtain at least one expected medication type; determine whether the at least one expected medication type corresponds to the at least one medication type associated with the identified at least one stored feature representation; and provide an indication of whether the at least one expected medication type corresponds to the at least one medication type associated with the identified at least one stored feature representation.

Clause 67: The computer program product of any of clauses 62-66, wherein the program instructions, when executed by the at least one processor, further cause the at least one processor to: control the ultrasonic flow sensor to generate the time-series corresponding to the flow of the at least one fluid through the fluid flow path of the ultrasonic flow sensor, wherein the ultrasonic flow sensor includes a flow tube that defines the fluid flow path of the ultrasonic flow sensor, a first piezoelectric sensor or transducer arranged at an upstream position of the flow tube, and a second piezoelectric sensor or transducer arranged at a downstream position of the flow tube, and wherein the first piezoelectric sensor or transducer and the second piezoelectric sensor or transducer of the ultrasonic flow sensor generate the time-series corresponding to the flow of the at least one fluid through the fluid flow path of the ultrasonic flow sensor.

Clause 68: The computer program product of any of clauses 62-67, wherein the program instructions, when executed by the at least one processor, further cause the at least one processor to: control at least one sensor to read medication information from a tag or label on a syringe, and wherein the medication information includes the at least one expected medication type.

Clause 69: The computer program product of any of clauses 62-68, wherein the program instructions, when executed by the at least one processor, further cause the at least one processor to: generate, using at least one machine learning model, based on the time-series, at least one of (i) a predicted start time associated with a start of the flow of the at least one fluid through the fluid flow path of the ultrasonic flow sensor, (ii) a predicted time period associated with the flow of the at least one fluid through the fluid flow path of the ultrasonic flow sensor, (iii) a predicted end time associated with an end of the flow of the at least one fluid through the fluid flow path of the ultrasonic flow sensor, or any combination thereof; and provide the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the end predicted time associated with the end of the flow of the at least one fluid through the fluid flow path of the ultrasonic flow sensor, or any combination thereof.

These and other features and characteristics of the present disclosure, as well as the methods of operation and functions of the related elements of structures and the combination of parts and economics of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosed subject matter.

### BRIEF DESCRIPTION OF THE DRAWINGS

Additional advantages and details are explained in greater detail below with reference to the non-limiting, exemplary embodiments that are illustrated in the accompanying schematic figures, in which:

FIG. 1A is a schematic diagram of a system for detecting fluid flow through ultrasonic flow sensors and/or identifying medication diversion, according to some non-limiting embodiments or aspects;

FIG. 1B is a perspective view of example components of a flow sensor system of the system for detecting fluid flow through ultrasonic flow sensors and/or identifying medication diversion of FIG. 1A, according to some non-limiting embodiments or aspects;

FIG. 1C is a cross-sectional view of example components of an ultrasonic flow sensor of a flow sensor system of the system for detecting fluid flow through ultrasonic flow sensors and/or identifying medication diversion of FIG. 1A, according to some non-limiting embodiments or aspects;

FIG. 2 is a schematic diagram of example components of one or more devices or systems of FIG. 1A, according to some non-limiting embodiments or aspects;

FIGS. 3A and 3B are a flow diagram of a method for detecting fluid flow through ultrasonic flow sensors and/or identifying medication diversion, according to some non-limiting embodiments or aspects; and

FIG. 4 is a diagram of an implementation of a method for detecting fluid flow through ultrasonic flow sensors and/or identifying medication diversion, according to some non-limiting embodiments or aspects; and

FIG. 5 is a diagram of an implementation of a method for detecting fluid flow through ultrasonic flow sensors and/or identifying medication diversion, according to some non-limiting embodiments or aspects.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the embodiments as they are oriented in the drawing figures. However, it is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects of the disclosed subject matter. Hence, specific dimensions and other physical characteristics related to the embodiments or aspects disclosed herein are not to be considered as limiting.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. In addition, reference to an action being “based on” a condition may refer to the action being “in response to” the condition. For example, the phrases “based on” and “in response to” may, in some non-limiting embodiments or aspects, refer to a condition for automatically triggering an action (e.g., a specific operation of an electronic device, such as a computing device, a processor, and/or the like).

As used herein, the term “communication” may refer to the reception, receipt, transmission, transfer, provision, and/or the like of data (e.g., information, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices. A computing device may also be a desktop computer or other form of non-mobile computer.

As used herein, the term “server” may refer to or include one or more computing devices that are operated by or facilitate communication and processing for multiple parties in a network environment, such as the Internet, although it will be appreciated that communication may be facilitated over one or more public or private network environments and that various other arrangements are possible. Further, multiple computing devices (e.g., servers, point-of-sale (POS) devices, mobile devices, etc.) directly or indirectly communicating in the network environment may constitute a “system.”

As used herein, the term “system” may refer to one or more computing devices or combinations of computing devices (e.g., processors, servers, client devices, software applications, components of such, and/or the like). Reference to “a device,” “a server,” “a processor,” and/or the like, as used herein, may refer to a previously-recited device, server, or processor that is recited as performing a previous step or function, a different device, server, or processor, and/or a combination of devices, servers, and/or processors. For example, as used in the specification and the claims, a first device, a first server, or a first processor that is recited as performing a first step or a first function may refer to the same or different device, server, or processor recited as performing a second step or a second function.

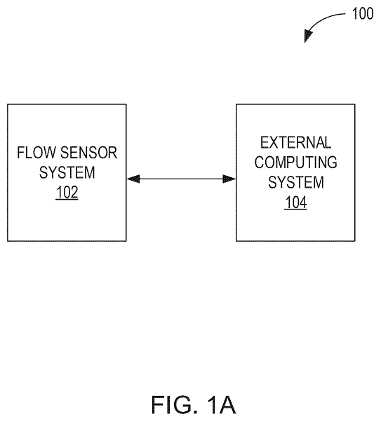

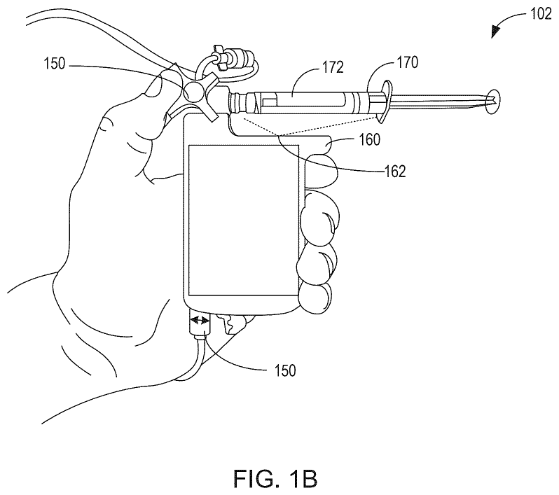

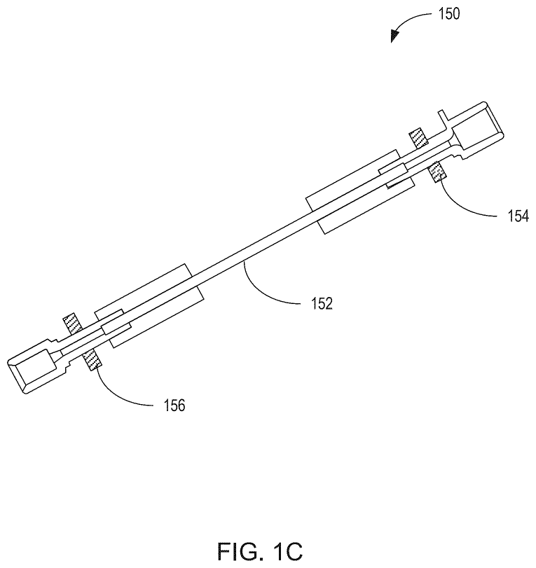

Referring now to FIG. 1A, shown is a schematic diagram of a system for detecting fluid flow through ultrasonic flow sensors and/or identifying medication diversion, according to some non-limiting embodiments or aspects. As shown in FIG. 1A, system 100 may include flow sensor system 102 and/or external computing system 104. Systems and/or devices of system 100 can interconnect via wired connections, wireless connections, or a combination of wired and wireless connections.

Flow sensor system 102 may include one or more devices capable of receiving information and/or data from external computing system 104 and/or communicating information and/or data to external computing system 104. For example, flow sensor system 102 may include one or more computing systems including one or more processors (e.g., one or more computing devices, one or more mobile computing devices, one or more digital signal processors (DSPs), etc.). In some non-limiting embodiments or aspects, flow sensor system 102 may include the flow sensor system in U.S. Patent Application Publication No. 2021/0231471.

Referring also to FIG. 1B, FIG. 1B is a perspective view of example components of a flow sensor system of the system for detecting fluid flow through ultrasonic flow sensors and/or identifying medication diversion of FIG. 1A, according to some non-limiting embodiments or aspects. As shown in FIG. 1B, flow sensor system 102 may include ultrasonic flow sensor 150 and/or base 160. For example, ultrasonic flow sensor 150 may be configured to be removably, physically, and/or electrically connected to base 160. Syringe 170 may be configured to physically connect to ultrasonic flow sensor 150 (e.g., via a fluid injection port, etc.). Syringe 170 may include tag or label 172, which may include a wireless communication tag (e.g., a near field communication (NFC) tag, a radio frequency identifier (RFID) tag, etc.) embedded in tag or label 172, a barcode, a quick read (QR) code, an AprilTag, or the like. Base 160 may include at least one sensor 162 configured to read and/or decode medication information from tag or label 172 on syringe 170. The medication information may include at least one expected medication type associated with at least one medication contained in syringe 170, such as a medication identifier (e.g., a unique medication identifier associated with the medication contained in syringe 170, etc.). For example, the at least one sensor 162 of base 160 may include one or more computing devices, chips, contactless transmitters, contactless transceivers, NFC transmitters/receivers, RFID transmitters/receivers, contact based transmitters/receivers, optical sensors or scanners, barcode readers, or the like that are configured to read and/or decode the medication information stored or encapsulated in tag or label 172.

Referring also to FIG. 1C, FIG. 1C is a cross-sectional view of components of an ultrasonic flow sensor of a flow sensor system of the system for detecting fluid flow through ultrasonic flow sensors and/or identifying medication diversion of FIG. 1A, according to some non-limiting embodiments or aspects. As shown in FIG. 1C, ultrasonic flow sensor 150 may include flow tube 152 that defines a fluid flow path of ultrasonic flow sensor 150, first piezoelectric sensor or transducer 154 arranged at an upstream position of flow tube 152, and/or second piezoelectric sensor or transducer 156 arranged at a downstream position of flow tube 152.

Ultrasonic flow sensor 150 may be configured to generate a time-series corresponding to (e.g., corresponding to, representative of, quantifying, measuring, etc.) a flow of at least one fluid through a fluid flow path of ultrasonic flow sensor 150. For example, first piezoelectric sensor or transducer 154 and second piezoelectric sensor or transducer 156 may be configured to generate the time-series corresponding to the flow of the at least one fluid through the fluid flow path of ultrasonic flow sensor 150. As an example, first piezoelectric sensor or transducer 154 and second piezoelectric sensor or transducer 156 may each be configured to operate as both an ultrasonic transmitter and an ultrasonic receiver. In such an example, ultrasonic flow sensor 150 may be configured to operate by alternately transmitting and receiving a burst of ultrasound between the two transducers by measuring the transit time that it takes for sound to travel between the two transducers in both directions. The difference in the transit time (e.g., A time, etc.) measured may be directly proportional to a velocity of the fluid in the fluid flow path. A plurality of differences in transit time (e.g., a plurality of A times, etc.) may be represented as a time-series that includes at least one of a plurality of amplitudes at a plurality of time points, a plurality of phases at the plurality of time points, or any combination thereof. Ultrasonic flow sensor 150 may be configured to continually generate and provide the time-series corresponding to the flow of at least one fluid through a fluid flow path of ultrasonic flow sensor 150 during the flow of at least one fluid through a fluid flow path of ultrasonic flow sensor 150 (e.g., as the flow of at least one fluid through a fluid flow path of ultrasonic flow sensor 150 occurs and progresses, etc.).

External computing system 104 may include one or more devices capable of receiving information and/or data from flow sensor system 102 and/or communicating information and/or data to flow sensor system 102. For example, external computing system 104 may include one or more computing systems including one or more processors (e.g., one or more computing devices, one or more mobile computing devices, one or more servers, etc.). In some non-limiting embodiments or aspects, external computing system 104 includes a nurse station in a hospital, a hospital information system (HIS), an electronic medical records (EMR) system, a radiology information system (RIS), a picture archiving and communication system (PACS), a laboratory information system (LIS), a smart phone, a tablet computer, any combination thereof, and/or the like.

The number and arrangement of systems and devices shown in FIGS. 1A-IC are provided as an example. There may be additional systems or devices, fewer systems or devices, different systems or devices, or differently arranged systems or devices than those shown in FIGS. 1A-1C. Furthermore, two or more systems or devices shown in FIGS. 1A-1C may be implemented within a single system or device, or a single system or device shown in FIGS. 1A-1C may be implemented as multiple, distributed systems or devices. Additionally or alternatively, a set of systems (e.g., one or more systems) or a set of devices (e.g., one or more devices) of system 100 may perform one or more functions described as being performed by another set of systems or another set of devices of system 100.

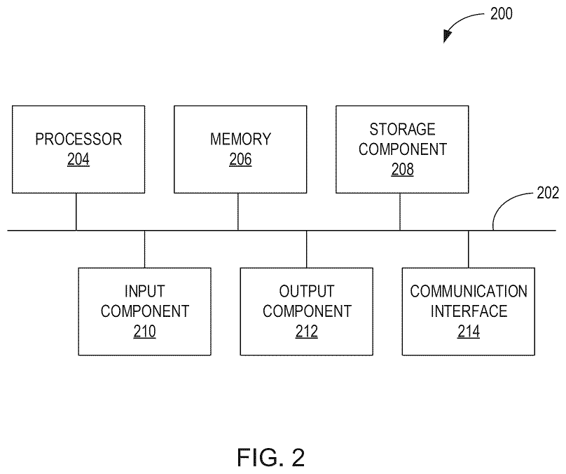

Referring now to FIG. 2, shown is a diagram of example components of a device 200 according to non-limiting embodiments. Device 200 may correspond to flow sensor system 102 and/or external computing system 104 in FIG. 1A, as an example. In some non-limiting embodiments, such systems or devices may include at least one device 200 and/or at least one component of device 200. The number and arrangement of components shown are provided as an example. In some non-limiting embodiments, device 200 may include additional components, fewer components, different components, or differently arranged components than those shown. Additionally, or alternatively, a set of components (e.g., one or more components) of device 200 may perform one or more functions described as being performed by another set of components of device 200.

As shown in FIG. 2, device 200 may include a bus 202, a processor 204, memory 206, a storage component 208, an input component 210, an output component 212, and a communication interface 214. Bus 202 may include a component that permits communication among the components of device 200. In some non-limiting embodiments, processor 204 may be implemented in hardware, firmware, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), or any processing component (e.g., a field-programmable gate array (FPGA), an application-specific integrated circuit (ASIC), etc.) that can be programmed to perform a function. Memory 206 may include random access memory (RAM), read only memory (ROM), or another type of dynamic or static storage device (e.g., flash memory, magnetic memory, optical memory, etc.) that stores information and/or instructions for use by processor 204.

With continued reference to FIG. 2, storage component 208 may store information and/or software related to the operation and use of device 200. For example, storage component 208 may include a hard disk (e.g., a magnetic disk, an optical disk, a magneto-optic disk, a solid-state disk, etc.) or another type of computer-readable medium. Input component 210 may include a component that permits device 200 to receive information, such as via user input (e.g., a touch screen display, a keyboard, a keypad, a mouse, a button, a switch, a microphone, etc.). Additionally, or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.). Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, or the like.

Device 200 may perform one or more processes described herein. Device 200 may perform these processes based on processor 204 executing software instructions stored by a computer-readable medium, such as memory 206 or storage component 208. A computer-readable medium may include any non-transitory memory device. A memory device includes memory space located inside of a single physical storage device or memory space spread across multiple physical storage devices. Software instructions may be read into memory 206 and/or storage component 208 from another computer-readable medium or from another device via communication interface 214. When executed, software instructions stored in memory 206 or storage component 208 may cause processor 204 to perform one or more processes described herein. Additionally, or alternatively, hardwired circuitry may be used in place of or in combination with software instructions to perform one or more processes described herein. Thus, embodiments described herein are not limited to any specific combination of hardware circuitry and software. The term “configured to,” as used herein, may refer to a specific arrangement of software, device(s), or hardware for performing or enabling one or more of the innovative functions (e.g., actions, processes, steps of a process, or the like) described herein. For example, “a processor configured to” may refer to a processor that executes specific software instructions (e.g., program code) that cause the processor to perform one or more functions related to fluid flow detection and/or identification.

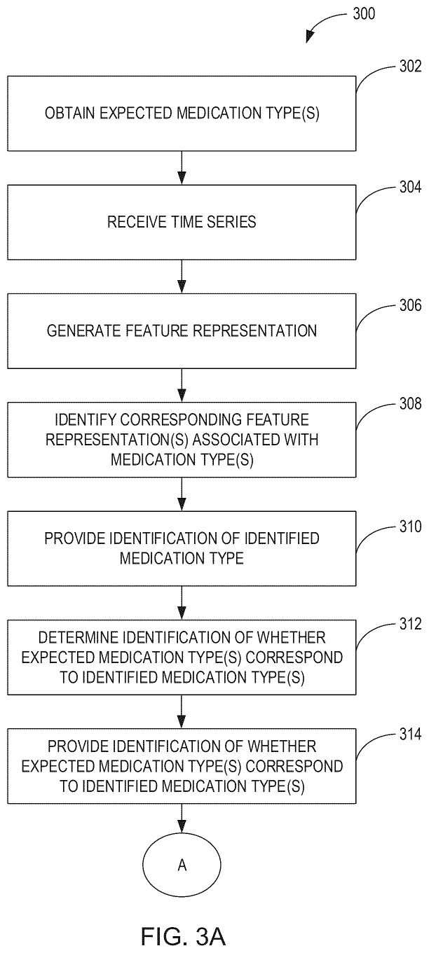

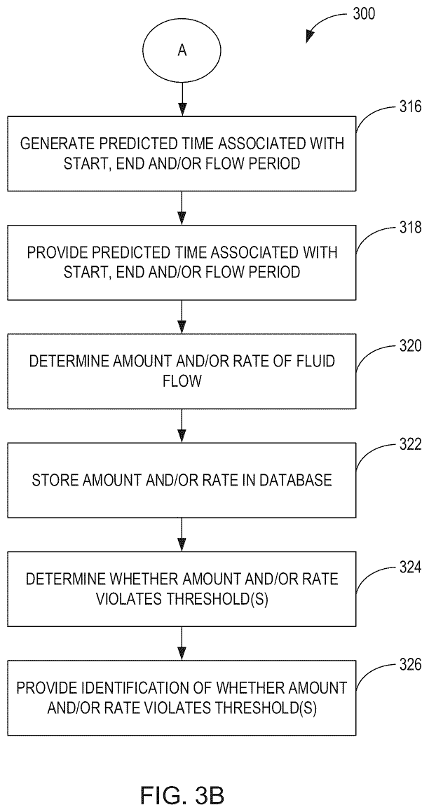

Referring now to FIGS. 3A and 3B, shown is a flow diagram for a method 300 for detecting fluid flow through ultrasonic flow sensors and/or identifying medication diversion, according to some non-limiting embodiments or aspects. The steps shown in FIGS. 3A and 3B are for example purposes only. It will be appreciated that additional, fewer, different, or a different order of steps may be used in some non-limiting embodiments or aspects. In some non-limiting embodiments or aspects, a step may be automatically performed in response to performance or completion of a prior step. The method 300 may be implemented in whole or in part by one or more of the systems or devices shown in FIGS. 1A, 1B, 1C, and 2.

As shown in FIG. 3A, at step 302, method 300 includes obtaining at least one expected medication type. For example, flow sensor system 102 and/or external computing system 104 may obtain at least one expected medication type. As an example, flow sensor system 102 and/or external computing system 104 may receive the at least one expected medication type (e.g., at least one medication identifier associated therewith, etc.) via user input to a user interface (e.g., via input component 210, etc.) and/or via the at least one sensor 162 of flow sensor system 102 reading and/or decoding medication information including the medication identifier of the at least one expected medication type from tag or label 172 on syringe 170, for example, when syringe 170 is connected to ultrasonic flow sensor 150. In such an example, different medication types (e.g., different drugs, different concentrations of a same drug, etc.) may be associated with different medication identifiers.

As shown in FIG. 3A, at step 304, method 300 includes receiving a time-series generated by the ultrasonic flow sensor corresponding to a flow of at least one fluid through a fluid flow path of the ultrasonic flow sensor. For example, flow sensor system 102 and/or external computing system 104 may receive a time-series generated by ultrasonic flow sensor 150 corresponding to (e.g., corresponding to, representative of, quantifying, measuring, etc.) a flow of at least one fluid through a fluid flow path of ultrasonic flow sensor 150. As an example, ultrasonic flow sensor 150 may generate the time-series during the flow of the at least one fluid through the fluid flow path of ultrasonic flow sensor 150. The time-series may include at least one of a plurality of amplitudes at a plurality of time points, a plurality of phases at the plurality of time points, or any combination thereof.

In some non-limiting embodiments or aspects, the at least one fluid includes a sequence of fluids. For example, the time-series may be generated by ultrasonic flow sensor 150 corresponding to (e.g., corresponding to, representative of, quantifying, measuring, etc.) a sequence of flows of the sequence of fluids through the fluid flow path of ultrasonic flow sensor 150. In some non-limiting embodiments or aspects, the time-series is further generated by ultrasonic flow sensor 150 corresponding to (e.g., corresponding to, representative of, quantifying, measuring, etc.) at least one period of no fluid flow through the fluid flow path of ultrasonic flow sensor 150. For example, the at least one period of no fluid flow through the fluid flow path of the ultrasonic sensor occurs before the flow of the fluid of the sequence of fluids through the fluid flow path of the ultrasonic sensor. As an example, the at least one period of no fluid flow through the fluid flow path of the ultrasonic sensor occurs after a flow of a fluid of the sequence of fluids through the fluid flow path of the ultrasonic sensor and before a flow of a next fluid of the sequence of fluids through the fluid flow path of the ultrasonic flow sensor. In such an example, the sequence of fluids may at least one saline bolus and at least one medication bolus.

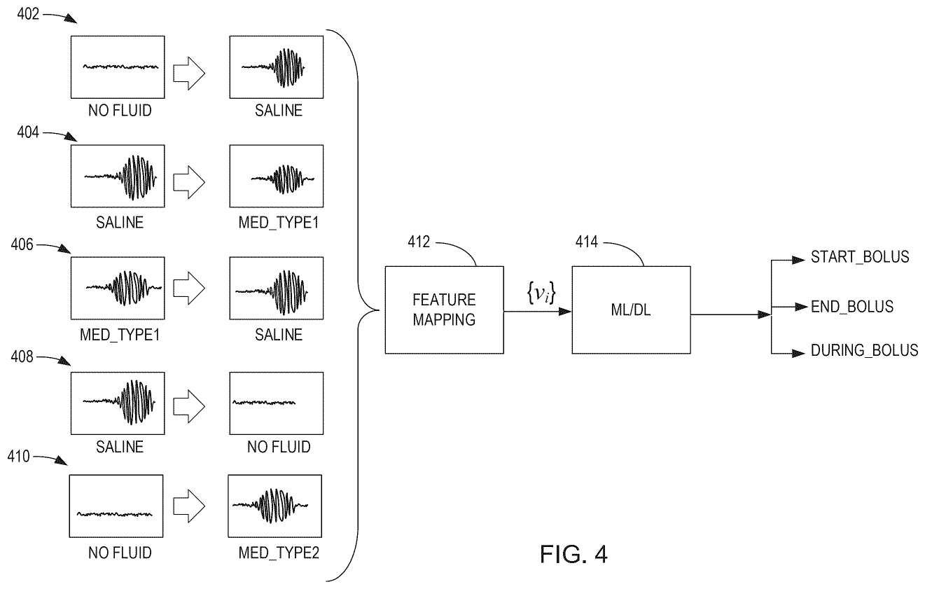

For example, and referring now to FIG. 4, FIG. 4 is a diagram of an implementation 400 of the method 300 for detecting fluid flow through ultrasonic flow sensors and/or identifying medication diversion, according to some non-limiting embodiments or aspects. As shown in FIG. 4, ultrasonic flow sensor 150 may generate a time-series corresponding to a sequence of flows of a sequence of fluids through the fluid flow path of ultrasonic flow sensor 150 that may include, a period of no fluid flow followed by a flow of a saline fluid (e.g., a saline bolus, etc.) at reference number 402, the flow of the saline fluid (e.g., the saline bolus, etc.) followed by a flow of a first medication type (e.g., a first medication bolus, etc.) at reference number 404, the flow of the first medication type (e.g., the first medication bolus, etc.) followed by a flow of a further saline fluid (e.g., a further saline bolus, etc.) at reference number 406, the flow of the further saline fluid (e.g., the further saline bolus, etc.) followed by a further period of no fluid flow at reference number 408, and the further period of no fluid flow followed by a flow of a second medication type (e.g., a second medication bolus, etc.) at reference number 410. In such an example, ultrasonic flow sensor 150 may continually generate the time-series in real-time (e.g., during the fluid flows and/or periods of no fluid flow occur, etc.). For example, flow sensor system 102 and/or external computing system 104 may continually receive the time-series in real-time as the time-series is generated by ultrasonic flow sensor 150.

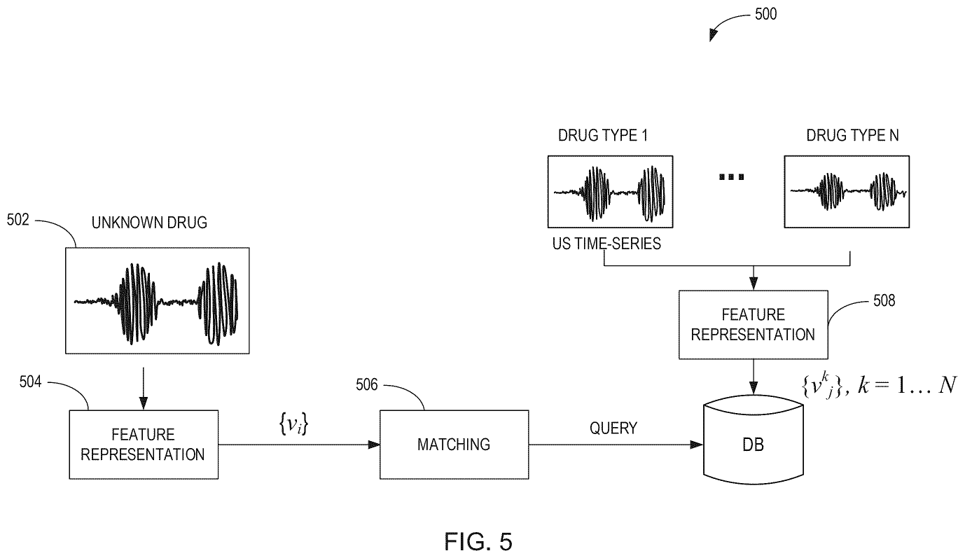

As an example, and referring now to FIG. 5, FIG. 5 is a diagram of an implementation 500 of the method 300 for detecting fluid flow through ultrasonic flow sensors and/or identifying medication diversion, according to some non-limiting embodiments or aspects. As shown in FIG. 5, at reference number 502, ultrasonic flow sensor 150 may generate a time-series corresponding to a flow of at least one fluid through a fluid flow path of ultrasonic flow sensor 150 (e.g., a single flow of a single fluid, a plurality of flows of a plurality of fluids, a sequence of flows of a sequence of fluids, etc.). In such an example, ultrasonic flow sensor 150 may continually generate the time-series in real-time (e.g., during the fluid flow, etc.). For example, flow sensor system 102 and/or external computing system 104 may continually receive the time-series in real-time as the time-series is generated by ultrasonic flow sensor 150.

As shown in FIG. 3A, at step 306, method 300 includes generating, using at least one feature extraction algorithm, based on the time-series including the plurality of amplitudes at the plurality of time points, the plurality of phases at the plurality of time points, or any combination thereof, a feature representation associated with the time series. For example, flow sensor system 102 and/or external computing system 104 may generate, using at least one feature extraction algorithm, based on the time-series including the plurality of amplitudes at the plurality of time points, the plurality of phases at the plurality of time points, or any combination thereof, a feature representation associated with the time series. As an example, flow sensor system 102 and/or external computing system 104 may provide, as input to the at least one feature extraction algorithm, the time-series, and receive, as output from the at least one feature extraction algorithm, a feature representation of the time-series that includes a feature representation (e.g., a feature vector representation, a high-dimensional feature vector representation, etc.) of the amplitude and/or phase at each time point of the time-series (e.g., a statistical feature at each time point of the time-series, such as a mean, an average, a standard deviation, and/or the like, etc.).

For example, and referring again to FIG. 4, at reference number 412, flow sensor system 102 and/or external computing system 104 may continually provide in real-time, as input to the at least one feature extraction algorithm, the time-series corresponding to the sequence of flows of the sequence of fluids through the fluid flow path of ultrasonic flow sensor 150 as the time-series is continually received in real-time from ultrasonic flow sensor 150, and continually receive in real-time, as output from the at least one feature extraction algorithm, the feature representation ({vi}) of the time-series that includes the feature representation of the amplitude and/or phase at each time point of the time-series.

As an example, and referring again to FIG. 5, at reference number 404, flow sensor system 102 and/or external computing system 104 may continually provide in real-time, as input to the at least one feature extraction algorithm, the time-series corresponding to the flow of at least one fluid through a fluid flow path of ultrasonic flow sensor 150 as the time-series is continually received in real-time from ultrasonic flow sensor 150, and continually receive in real-time, as output from the at least one feature extraction algorithm, the feature representation of the time-series that includes the feature representation of the amplitude and/or phase at each time point of the time-series.

As shown in FIG. 3A, at step 308, method 300 includes identifying, based on the time series generated by the ultrasonic flow sensor corresponding to the flow of the at least one fluid through the fluid flow path of the ultrasonic flow sensor, in a database including a plurality of stored feature representations associated with a plurality of medication types, at least one stored feature representation of the plurality of stored feature representations, wherein the at least one stored feature vector representation is associated with at least one medication type. For example, flow sensor system 102 and/or external computing system 104 may identify, based on the time series generated by ultrasonic flow sensor 150 corresponding to the flow of the at least one fluid through the fluid flow path of ultrasonic flow sensor 150, in a database including a plurality of stored feature vector representations (e.g., a plurality of feature vector representations, a plurality of high-dimensional feature vector representations, etc.) associated with a plurality of medication types, at least one stored feature representation (e.g., at least one stored feature vector representation, at least one high-dimensional feature vector representation, etc.) of the plurality of stored feature representations. The at least one stored feature representation may be associated with at least one medication type (e.g., with at least one medication identifier, etc.). As an example, flow sensor system 102 and/or external computing system 104 may identify, in the database including the plurality of stored feature representations associated with the plurality of medication types, the at least one stored feature representation of the plurality of stored feature representations that corresponds to the feature representation associated with the time-series.

In some non-limiting embodiments or aspects, flow sensor system 102 and/or external computing system 104 may use a closed form expression algorithm or technique (e.g., a cross-correlation algorithm or technique, a sum of squared errors algorithm or technique, etc.) to identify, in the database including the plurality of stored feature representations associated with the plurality of medication types, the at least one stored feature representation of the plurality of stored feature representations that corresponds to the feature representation associated with the time-series. For example, flow sensor system 102 and/or external computing system 104 may use a closed form expression algorithm or technique that measures a similarity or match between two feature representations to query the database to identify the at least one stored feature representation of the plurality of stored feature representations that corresponds to (e.g. is closer to, etc.) the feature representation associated with the time-series.

In some non-limiting embodiments or aspects, flow sensor system 102 and/or external computing system 104 may use one or more machine learning models to identify, in the database including the plurality of stored feature representations associated with the plurality of medication types, the at least one stored feature representation of the plurality of stored feature representations that corresponds to the feature representation associated with the time-series. For example, flow sensor system 102 and/or external computing system 104 may use one or more machine learning models that measure a similarity or match between two feature representations to query the database to identify the at least one stored feature representation of the plurality of stored feature representations that corresponds to (e.g. is closer to, etc.) the feature representation associated with the time-series.

The one or more machine learning models (e.g., a neural network, a recurrent neural network, a convolutional neural network, etc.) may be trained using machine learning or other artificial intelligence techniques to accept, as input, the time-series (e.g., the feature representation associated with the time-series, etc.) and the plurality of stored time series (e.g., the plurality of stored feature representations associated with the plurality of medication types, etc.) and provide, as output, the at least one stored feature representation of the plurality of stored feature representations and/or a medication identifier associated therewith that corresponds to the feature representation associated with the time-series. For example, flow sensor system 102 and/or external computing system 104 may generate, using the one or more machine learning models, based on the time-series, a prediction of the at least one stored feature representation of the plurality of stored feature representations that corresponds to (e.g. is closer to, etc.) the feature representation associated with the time-series by: providing, as input to the one or more machine learning models, the feature representation associated with the time series; and receiving, as output from the one or more machine learning models, the at least one stored feature representation of the plurality of stored feature representations and/or a medication identifier associated therewith that corresponds to the feature representation associated with the time-series.

Referring again to FIG. 5, at reference number 506, flow sensor system 102 and/or external computing system 104 may query a database including a plurality of stored feature representations associated with a plurality of medication types to identify at least one stored feature representation of the plurality of stored feature representations that corresponds to the time series generated by ultrasonic flow sensor 150 corresponding to the flow of the at least one fluid through the fluid flow path of ultrasonic flow sensor 150. As an example, at reference number 508, flow sensor system 102 and/or external computing system 104 may generate (e.g., offline or in advance of performing steps 302-308 of FIG. 3A, etc.) the plurality of stored feature representations associated with the plurality of medication types (e.g., a database of drug inventory created offline or in advance that captures and stores a signature feature representation per drug, etc.) in a same or similar manner as described herein with respect to steps 304 and 306 of FIG. 3A. In such an example, the plurality of stored feature representations associated with the plurality of medication types may have a same dimension as the feature representation associated with the time-series generated by ultrasonic flow sensor 150 corresponding to the flow of the at least one fluid through the fluid flow path of ultrasonic flow sensor 150. For example, flow sensor system 102 and/or external computing system 104 may receive, for each medication type or drug to be stored in the database, a time-series generated by ultrasonic flow sensor 150 corresponding to a flow of that medication type or drug through the fluid flow path of ultrasonic flow sensor 150, generate, using the at least one feature extraction algorithm, based on that time-series, a feature representation associated with that time-series and medication or drug, and/or store that feature representation in the database.

As shown in FIG. 3A, at step 310, method 300 includes providing an indication of the at least one medication type. For example, flow sensor system 102 and/or external computing system 104 may provide an indication of the at least one medication type. The indication may be a human perceivable output indicating the identified at least one medication type (e.g., provided via a speaker and/or a display of a reusable base of the flow sensor system in U.S. Patent Application Publication No. 2021/0231471, etc.). In some implementations, providing the indication may include storing a value in a location of a storage device for subsequent retrieval, transmitting a value directly to the recipient via at least one wired or wireless communication medium, transmitting or storing a reference to a value, and the like. The providing at step 310 may additionally or alternatively include encoding, decoding, encrypting, decrypting, validating, verifying, and the like via a hardware element.

In this way, non-limiting embodiments or aspects of the present disclosure may automatically identify drug diversion at a time of injection without relying on reading a tag or label (e.g., a RFID tag, a barcode, etc.).

As shown in FIG. 3A, at step 312, method 300 includes determining whether the at least one expected medication type corresponds to the at least one medication type associated with the identified at least one stored feature representation. For example, flow sensor system 102 and/or external computing system 104 may determine whether the at least one expected medication type corresponds to the at least one medication type associated with the identified at least one stored feature representation. As an example, flow sensor system 102 and/or external computing system 104 may determine whether the at least one expected medication type is a same medication or a same type of medication as the at least one medication type associated with the identified at least one stored feature representation.

As shown in FIG. 3A, at step 314, method 300 includes providing an indication of whether the at least one expected medication type corresponds to the at least one medication type associated with the identified at least one stored feature representation. For example, flow sensor system 102 and/or external computing system 104 may provide an indication of whether the at least one expected medication type corresponds to the at least one medication type associated with the identified at least one stored feature representation. The indication may be a human perceivable output indicating whether the at least one expected medication type corresponds to the at least one medication type associated with the identified at least one stored feature representation (e.g., provided via a speaker and/or a display of a reusable base of the flow sensor system in U.S. Patent Application Publication No. 2021/0231471, etc.). In some implementations, providing the indication may include storing a value in a location of a storage device for subsequent retrieval, transmitting a value directly to the recipient via at least one wired or wireless communication medium, transmitting or storing a reference to a value, and the like. The providing at step 310 may additionally or alternatively include encoding, decoding, encrypting, decrypting, validating, verifying, and the like via a hardware element.

In some non-limiting embodiments or aspects, flow sensor system 102 and/or external computing system 104 may provide the indication of whether the at least one expected medication type corresponds to the at least one medication type associated with the identified at least one stored feature representation by controlling a user interface associated with ultrasonic flow sensor 150 (e.g., a speaker and/or a display of a reusable base of the flow sensor system in U.S. Patent Application Publication No. 2021/0231471, etc.) to output at least one of an audio output, a visual output, or any combination thereof associated with whether the at least one expected medication type corresponds to the at least one medication type associated with the identified at least one stored feature representation. For example, a user may purposely or inadvertently divert medication by delivering a different medication than a medication identified by the user or a scanned tag or label. Accordingly, flow sensor system 102 and/or external computing system 104 may alert the user (e.g., a caregiver, a nurse, trainee, etc.) and/or a supervisor of the user when the at least one expected medication type does not correspond to a medication delivered or being delivered (e.g., to the at least one medication type associated with the identified at least one stored feature representation, etc.), thereby reducing medication diversion and/or delivery errors.

In some non-limiting embodiments or aspects, flow sensor system 102 and/or external computing system 104 may provide the indication of whether the at least one of the amount of the at least one fluid that flowed through the fluid flow path of ultrasonic flow sensor 150, the rate at which the at least one fluid flowed or is flowing through the fluid flow path of ultrasonic flow sensor 150, or any combination thereof, violates the at least one of the threshold amount associated with at least one fluid, the threshold rate associated with the at least one fluid, or any combination thereof in association with patient data associated with a patient, procedure data associated with a patient procedure associated with the patient, caregiver data associated with a caregiver (e.g., a nurse, a doctor, etc.), any combination thereof, or the like.

In this way, because different medications may be associated with different signal patterns, non-limiting embodiments or aspects of the present disclosure may use the signal pattern of the time-series to identify a type of medication delivered or being delivered during the flow of the at least one fluid through the fluid flow path of an ultrasonic flow sensor 150.

As shown in FIG. 3B, at step 316, method 300 includes generating, using at least one machine learning model, based on the time-series, at least one of (i) a predicted start time associated with a start of the flow of the at least one fluid through the fluid flow path of the ultrasonic flow sensor, (ii) a predicted time period associated with the flow of the at least one fluid through the fluid flow path of the ultrasonic flow sensor, (iii) a predicted end time associated with an end of the flow of the at least one fluid through the fluid flow path of the ultrasonic flow sensor, or any combination thereof. For example, flow sensor system 102 and/or external computing system 104 may generate, using at least one machine learning model, based on the time-series, at least one of (i) a predicted start time associated with a start of the flow of the at least one fluid through the fluid flow path of ultrasonic flow sensor 150, (ii) a predicted time period associated with the flow of the at least one fluid through the fluid flow path of ultrasonic flow sensor 150, (iii) a predicted end time associated with an end of the flow of the at least one fluid through the fluid flow path of ultrasonic flow sensor 150, or any combination thereof.

In some non-limiting embodiments or aspects, flow sensor system 102 and/or external computing system 104 may further use the at least one expected medication type and/or the at least one identified medication type to generate the at least one of (i) the predicted start time associated with the start of the flow of the at least one fluid through the fluid flow path of ultrasonic flow sensor 150, (ii) the predicted time period associated with the flow of the at least one fluid through the fluid flow path of ultrasonic flow sensor 150, (iii) the predicted end time associated with the end of the flow of the at least one fluid through the fluid flow path of ultrasonic flow sensor 150, or any combination thereof. For example, flow sensor system 102 and/or external computing system 104 may generate, using the at least one machine learning model, based on the time-series, the at least one expected medication type, and/or the at least one identified medication type, the at least one of (i) the predicted start time associated with the start of the flow of the at least one fluid through the fluid flow path of ultrasonic flow sensor 150, (ii) the predicted time period associated with the flow of the at least one fluid through the fluid flow path of ultrasonic flow sensor 150, (iii) the predicted end time associated with the end of the flow of the at least one fluid through the fluid flow path of ultrasonic flow sensor 150, or any combination thereof. As an example, knowing a type of the medication flowing through the fluid flow path of the ultrasonic flow sensor 150 by providing the at least one expected medication type and/or the at least one identified medication type as additional features to the at least one machine learning model may help improve an accuracy of the predictions of the at least one machine learning models for the predicted start time, predicted time period, and/or prediction end time of the fluid flow.

The at least one machine learning model (e.g., a neural network, a recurrent neural network, a convolutional neural network, etc.) may be trained using machine learning or other artificial intelligence techniques to accept, as input, the time-series, the at least one expected medication type, and/or the at least one identified medication type (and/or feature representations thereof) and provide, as output, the at least one of (i) the predicted start time associated with the start of the flow of the at least one fluid through the fluid flow path of ultrasonic flow sensor 150, (ii) the predicted time period associated with the flow of the at least one fluid through the fluid flow path of the ultrasonic flow sensor 150, (iii) the predicted end time associated with an end of the flow of the at least one fluid through the fluid flow path of ultrasonic flow sensor 150, or any combination thereof associated with the flow of the at least one fluid through the fluid flow path of ultrasonic flow sensor 150. For example, flow sensor system 102 and/or external computing system 104 may generate, using the at least one machine learning model, based on the time-series, the at least one of (i) the predicted start time associated with the start of the flow of the at least one fluid through the fluid flow path of ultrasonic flow sensor 150, (ii) the predicted time period associated with the flow of the at least one fluid through the fluid flow path of ultrasonic flow sensor 150, (iii) the predicted end time associated with the end of the flow of the at least one fluid through the fluid flow path of ultrasonic flow sensor 150, or any combination thereof, by: providing, as input to the at least one machine learning model, the feature representation associated with the time series, the at least one expected medication type, and/or the at least one identified medication type; and receiving, as output from the at least one machine learning model, the at least one of (i) the predicted start time associated with the start of the flow of the at least one fluid through the fluid flow path of ultrasonic flow sensor 150, (ii) the predicted time period associated with the flow of the at least one fluid through the fluid flow path of ultrasonic flow sensor 150, (iii) the predicted end time associated with the end of the flow of the at least one fluid through the fluid flow path of ultrasonic flow sensor 150, or any combination thereof.

As described herein, in some non-limiting embodiments or aspects, the at least one fluid includes a sequence of fluids. For example, the time-series may be generated by ultrasonic flow sensor 150 corresponding to (e.g., corresponding to, representative of, quantifying, measuring, etc.) a sequence of flows of the sequence of fluids through the fluid flow path of ultrasonic flow sensor 150. As an example, the at least one of (i) the predicted start time associated with the start of the flow of the at least one fluid through the fluid flow path of ultrasonic flow sensor 150, (ii) the predicted time period associated with the flow of the at least one fluid through the fluid flow path of ultrasonic flow sensor 150, (iii) the predicted end time associated with the end of the flow of the at least one fluid through the fluid flow path of ultrasonic flow sensor, or any combination thereof may include: at least one of (a) one or more predicted start times associated with one or more starts of one or more flows of one or more fluids of the sequence of fluids through the fluid flow path of ultrasonic flow sensor 150 (e.g., a plurality of predicted start times associated with a plurality of starts of a plurality of flows of a plurality of fluids of the sequence of fluids through the fluid flow path ultrasonic flow sensor 150, etc.), (b) one or more predicted time periods associated with the one or more flows of the one or more fluids of the sequence of fluids through the fluid flow path of ultrasonic flow sensor 150 (e.g., a plurality of predicted time periods associated with the plurality of flows of the plurality of fluids of the sequence of fluids through the fluid flow path ultrasonic flow sensor 150, etc.), (c) one or more predicted end times associated with one or more ends of the one or more flows of the one or more fluids of the sequence of fluids through the fluid flow path of ultrasonic flow sensor 150 (e.g., a plurality of predicted end times associated with a plurality of ends of the plurality of flows of the plurality of fluids of the sequence of fluids through the fluid flow path ultrasonic flow sensor 150, etc.), or any combination thereof.

As described herein, in some non-limiting embodiments or aspects, the time-series is further generated by ultrasonic flow sensor 150 corresponding to at least one period of no fluid flow through the fluid flow path of ultrasonic flow sensor 150. For example, flow sensor system 102 and/or external computing system 104 may generate using the at least one machine learning model, based on the time-series, at least one of (x) a predicted no flow start time associated with a start of the at least one period of no fluid flow through the fluid flow path of ultrasonic flow sensor 150, (y) a predicted no flow time period associated with the at least one period of no fluid flow through the fluid flow path of ultrasonic flow sensor 150, (z) a predicted no flow end time associated with an end of the at least one period of no fluid flow through the fluid flow path of ultrasonic flow sensor 150, or any combination thereof. As an example, the at least one machine learning model (e.g., a neural network, a recurrent neural network, a convolutional neural network, etc.) may be trained using machine learning or other artificial intelligence techniques to accept, as input, the time-series, the at least one expected medication type, and/or the at least one identified medication type (and/or feature representations thereof) and provide, as output, the at least one of (x) the predicted no flow start time associated with the start of the at least one period of no fluid flow through the fluid flow path of ultrasonic flow sensor 150, (y) the predicted no flow time period associated with the at least one period of no fluid flow through the fluid flow path of ultrasonic flow sensor 150, (z) the predicted no flow end time associated with an end of the at least one period of no fluid flow through the fluid flow path of ultrasonic flow sensor 150, or any combination thereof.

For example, and referring again to FIG. 4, at reference number 414, flow sensor system 102 and/or external computing system 104 may continually provide in real-time, as input to the at least one machine learning model, the feature representation of the time-series that includes the feature representation of the amplitude and/or phase at each time point of the time-series, and continually receive in real-time, as output from the at least one machine learning model, predictions or indications of the start, the period of in progress, and/or the end and/or a type of medication administered for each of the period of no fluid flow, the flow of a saline fluid (e.g., the saline bolus, etc.), the flow of the first medication type (e.g., the first medication bolus, etc.), the flow of the further saline fluid (e.g., the further saline bolus, etc.), the further period of no fluid flow, and the flow of the second medication type (e.g., the second medication bolus, etc.).

In this way, non-limiting embodiments or aspects of the present disclosure may detect a change in medication administration (e.g., a start and an end of a saline bolus versus a start and an end of specific medication versus a start and an end of a period of no fluid flow, etc.) through a change in signal pattern (e.g., in amplitude, in phase, etc.) measured by an ultrasonic flow sensor that, even though the change in signal pattern may be subtle, is detectable and identifiable by a machine learning model. Accordingly, non-limiting embodiments or aspects of the present disclosure may provide for real-time (e.g., during fluid flow, immediately after fluid flow begins, immediately after fluid flow ends, during medication administration, immediately after medication administration begins, immediately after medication administration ends, etc.) detection of a start and an end of medication administration.

As shown in FIG. 3A, at step 318, method 300 includes providing the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the end predicted time associated with the end of the flow of the at least one fluid through the fluid flow path of the ultrasonic flow sensor, or any combination thereof. For example, flow sensor system 102 and/or external computing system 104 may provide the at least one of (i) the predicted start time associated with the start of the flow of the at least one fluid through the fluid flow path of ultrasonic flow sensor 150, (ii) the predicted time period associated with the flow of the at least one fluid through the fluid flow path of ultrasonic flow sensor 150, (iii) the end predicted time associated with the end of the flow of the at least one fluid through the fluid flow path of ultrasonic flow sensor 150, or any combination thereof. As an example, flow sensor system 102 and/or external computing system 104 may provide an indication associated with the at least one of (i) the predicted start time associated with the start of the flow of the at least one fluid through the fluid flow path of ultrasonic flow sensor 150, (ii) the predicted time period associated with the flow of the at least one fluid through the fluid flow path of ultrasonic flow sensor 150, (iii) the end predicted time associated with the end of the flow of the at least one fluid through the fluid flow path of ultrasonic flow sensor 150, or any combination thereof. The indication may be a human perceivable output indicating the at least one of (i) the predicted start time associated with the start of the flow of the at least one fluid through the fluid flow path of ultrasonic flow sensor 150, (ii) the predicted time period associated with the flow of the at least one fluid through the fluid flow path of ultrasonic flow sensor 150, (iii) the end predicted time associated with the end of the flow of the at least one fluid through the fluid flow path of ultrasonic flow sensor 150, or any combination thereof (e.g., provided via a speaker and/or a display of a reusable base of the flow sensor system in U.S. Patent Application Publication No. 2021/0231471, etc.). In some implementations, providing the indication may include storing a value in a location of a storage device for subsequent retrieval, transmitting a value directly to the recipient via at least one wired or wireless communication medium, transmitting or storing a reference to a value, and the like. The providing at step 308 may additionally or alternatively include encoding, decoding, encrypting, decrypting, validating, verifying, and the like via a hardware element.

In some non-limiting embodiments or aspects, flow sensor system 102 and/or external computing system 104 may provide the indication associated with the at least one of (i) the predicted start time associated with the start of the flow of the at least one fluid through the fluid flow path of ultrasonic flow sensor 150, (ii) the predicted time period associated with the flow of the at least one fluid through the fluid flow path of ultrasonic flow sensor 150, (iii) the end predicted time associated with the end of the flow of the at least one fluid through the fluid flow path of ultrasonic flow sensor 150, or any combination thereof, the indication associated with the at least one identified medication type, and/or the indication of whether the at least one expected medication type corresponds to the at least one identified medication type in association with patient data associated with a patient, procedure data associated with a patient procedure associated with the patient, caregiver data associated with a caregiver (e.g., a nurse, a doctor, etc.), any combination thereof, or the like. Patient data associated with a patient may include a patient identifier associated with the patient (e.g., a unique patient identifier, etc.), patient demographics (e.g., a name, an age, a sex, a weight, a height, a birthdate, an address, etc.), a list of medication allergies associated with the patient, a list of medication doses delivered, being delivered, and/or pending for delivery to the patient, any combination thereof, or the like. Procedure data may include a procedure identifier associated with the procedure (e.g., a unique procedure identifier, etc.), one or more medical devices associated with the procedure, a name of the procedure, a state of the procedure (e.g., scheduled for a future date and time, currently being performed, previously performed a previous date and time, etc.), a caregiver associated with the procedure, a patient associated with the procedure, any combination thereof, or the like. Caregiver data may include a caregiver identifier associated with the caregiver (e.g., a unique caregiver identifier, etc.), a name of the caregiver, any combination thereof, or the like.

As shown in FIG. 3B, at step 320, method 300 includes determining based on the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, at least one of an amount of the at least one fluid that flowed through the fluid flow path of the ultrasonic flow sensor, a rate at which the at least one fluid flowed or is flowing through the fluid flow path of the ultrasonic flow sensor, or any combination thereof. For example, flow sensor system 102 and/or external computing system 104 may determine, based on the at least one of (i) the predicted start time associated with the start of the flow of the at least one fluid through the fluid flow path of ultrasonic flow sensor 150, (ii) the predicted time period associated with the flow of the at least one fluid through the fluid flow path of ultrasonic flow sensor 150, (iii) the predicted end time associated with the end of the flow of the at least one fluid through the fluid flow path of ultrasonic flow sensor 150, or any combination thereof, at least one of an amount of the at least one fluid that flowed through the fluid flow path of ultrasonic flow sensor 150, a rate at which the at least one fluid flowed or is flowing through the fluid flow path of ultrasonic flow sensor 150, or any combination thereof. As an example, flow sensor system 102 and/or external computing system 104 may use the at least one of (i) the predicted start time associated with the start of the flow of the at least one fluid through the fluid flow path of ultrasonic flow sensor 150, (ii) the predicted time period associated with the flow of the at least one fluid through the fluid flow path of ultrasonic flow sensor 150, (iii) the predicted end time associated with the end of the flow of the at least one fluid through the fluid flow path of ultrasonic flow sensor 150, or any combination thereof, known dimensions of flow tube 152, and known equations for calculating the velocity of a fluid in a flow tube to calculate the at least one of the amount of the at least one fluid that flowed through the fluid flow path of ultrasonic flow sensor 150, the rate at which the at least one fluid flowed or is flowing through the fluid flow path of ultrasonic flow sensor 150, or any combination thereof. In such an example, flow sensor system 102 and/or external computing system 104 may provide the at least one of the amount of the at least one fluid that flowed through the fluid flow path of ultrasonic flow sensor 150, the rate at which the at least one fluid flowed or is flowing through the fluid flow path of ultrasonic flow sensor 150, or any combination thereof. In this way, non-limiting embodiments or aspects of the present disclosure may enable a more accurate measurement of the amount of the at least one fluid that flowed or is flowing through the fluid flow path of ultrasonic flow sensor 150, a more accurate measurement of the rate at which the at least one fluid flowed through the fluid flow path of ultrasonic flow sensor 150, or any combination thereof.

As shown in FIG. 3B at step 322, method 300 includes automatically storing, in a database, in association with a record of a patient, the at least one of the amount of the at least one fluid that flowed or is flowing through the fluid flow path of the ultrasonic flow sensor, the rate at which the at least one fluid flowed through the fluid flow path of the ultrasonic flow sensor, or any combination thereof. For example, flow sensor system 102 and/or external computing system 104 may automatically store, in a database, in association with a record of a patient, the at least one of the amount of the at least one fluid that flowed or is flowing through the fluid flow path of ultrasonic flow sensor 150, the rate at which the at least one fluid flowed through the fluid flow path of ultrasonic flow sensor 150, or any combination thereof. As an example, flow sensor system 102 and/or external computing system 104 may store, in the database, the at least one of the amount of the at least one fluid that flowed or is flowing through the fluid flow path of ultrasonic flow sensor 150, the rate at which the at least one fluid flowed through the fluid flow path of ultrasonic flow sensor 150, or any combination thereof in association with patient data associated with a patient, procedure data associated with a patient procedure associated with the patient, caregiver data associated with a caregiver (e.g., a nurse, a doctor, etc.), any combination thereof, or the like.

As shown in FIG. 3B at step 324, method 300 includes determining whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates at least one of a threshold amount associated with at least one fluid, a threshold rate associated with the at least one fluid, or any combination thereof. For example, flow sensor system 102 and/or external computing system 104 may determine whether the at least one of the amount of the at least one fluid that flowed through the fluid flow path of ultrasonic flow sensor 150, the rate at which the at least one fluid flowed or is flowing through the fluid flow path of ultrasonic flow sensor 150, or any combination thereof, violates at least one of a threshold amount associated with at least one fluid, a threshold rate associated with the at least one fluid, or any combination thereof.

As shown in FIG. 3B at step 326, method 300 includes providing an indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof. For example, flow sensor system 102 and/or external computing system 104 may provide an indication of whether the at least one of the amount of the at least one fluid that flowed through the fluid flow path of ultrasonic flow sensor 150, the rate at which the at least one fluid flowed or is flowing through the fluid flow path of ultrasonic flow sensor 150, or any combination thereof, violates the at least one of the threshold amount associated with at least one fluid, the threshold rate associated with the at least one fluid, or any combination thereof. The indication may be a human perceivable output indicating whether the at least one of the amount of the at least one fluid that flowed through the fluid flow path of ultrasonic flow sensor 150, the rate at which the at least one fluid flowed or is flowing through the fluid flow path of ultrasonic flow sensor 150, or any combination thereof, violates the at least one of the threshold amount associated with at least one fluid, the threshold rate associated with the at least one fluid, or any combination thereof (e.g., provided via a speaker and/or a display of a reusable base of the flow sensor system in U.S. Patent Application Publication No. 2021/0231471, etc.). In some implementations, providing the indication may include storing a value in a location of a storage device for subsequent retrieval, transmitting a value directly to the recipient via at least one wired or wireless communication medium, transmitting or storing a reference to a value, and the like. The providing at step 308 may additionally or alternatively include encoding, decoding, encrypting, decrypting, validating, verifying, and the like via a hardware element.

In some non-limiting embodiments or aspects, flow sensor system 102 and/or external computing system 104 may provide the indication of whether the at least one of the amount of the at least one fluid that flowed through the fluid flow path of ultrasonic flow sensor 150, the rate at which the at least one fluid flowed or is flowing through the fluid flow path of ultrasonic flow sensor 150, or any combination thereof, violates the at least one of the threshold amount associated with at least one fluid, the threshold rate associated with the at least one fluid, or any combination thereof, by controlling a user interface associated with ultrasonic flow sensor 150 (e.g., a speaker and/or a display of a reusable base of the flow sensor system in U.S. Patent Application Publication No. 2021/0231471, etc.) to output at least one of an audio output, a visual output, or any combination thereof associated with whether the at least one of the amount of the at least one fluid that flowed through the fluid flow path of ultrasonic flow sensor 150, the rate at which the at least one fluid flowed or is flowing through the fluid flow path of ultrasonic flow sensor 150, or any combination thereof, violates the at least one of the threshold amount associated with at least one fluid, the threshold rate associated with the at least one fluid, or any combination thereof. For example, a medication may be associated with thresholds that indicate that the medication should not be delivered at a rate greater than a threshold rate and/or in an amount greater than a threshold amount. Accordingly, flow sensor system 102 and/or external computing system 104 may alert a user (e.g., a caregiver, a nurse, trainee, etc.) when the rate at which the medication is being delivered and/or an amount of the medication delivered exceeds these thresholds, thereby reducing medication delivery errors to patients and/or improving training for medication delivery.

In some non-limiting embodiments or aspects, flow sensor system 102 and/or external computing system 104 may provide the indication of whether the at least one of the amount of the at least one fluid that flowed through the fluid flow path of ultrasonic flow sensor 150, the rate at which the at least one fluid flowed or is flowing through the fluid flow path of ultrasonic flow sensor 150, or any combination thereof, violates the at least one of the threshold amount associated with at least one fluid, the threshold rate associated with the at least one fluid, or any combination thereof in association with patient data associated with a patient, procedure data associated with a patient procedure associated with the patient, caregiver data associated with a caregiver (e.g., a nurse, a doctor, etc.), any combination thereof, or the like.

Although embodiments have been described in detail for the purpose of illustration, it is to be understood that such detail is solely for that purpose and that the disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

Aspects described include artificial intelligence or other operations whereby the system processes inputs and generates outputs with apparent intelligence. The artificial intelligence may be implemented in whole or in part by a model. A model may be implemented as a machine learning model. The learning may be supervised, unsupervised, reinforced, or a hybrid learning whereby multiple learning techniques are employed to generate the model. The learning may be performed as part of training. Training the model may include obtaining a set of training data and adjusting characteristics of the model to obtain a desired model output. For example, three characteristics may be associated with a desired item location. In such instance, the training may include receiving the three characteristics as inputs to the model and adjusting the characteristics of the model such that for each set of three characteristics, the output device state matches the desired device state associated with the historical data.

In some implementations, the training may be dynamic. For example, the system may update the model using a set of events. The detectable properties from the events may be used to adjust the model.

The model may be an equation, artificial neural network, recurrent neural network, convolutional neural network, decision tree, or other machine-readable artificial intelligence structure. The characteristics of the structure available for adjusting during training may vary based on the model selected. For example, if a neural network is the selected model, characteristics may include input elements, network layers, node density, node activation thresholds, weights between nodes, input or output value weights, or the like. If the model is implemented as an equation (e.g., regression), the characteristics may include weights for the input parameters, thresholds, or limits for evaluating an output value, or criterion for selecting from a set of equations.

Once a model is trained, retraining may be included to refine or update the model to reflect additional data or specific operational conditions. The retraining may be based on one or more signals detected by a device described herein or as part of a method described herein. Upon detection of the designated signals, the system may activate a training process to adjust the model as described.

Further examples of machine learning and modeling features which may be included in the embodiments discussed above are described in “A survey of machine learning for big data processing” by Qiu et al. in EURASIP Journal on Advances in Signal Processing (2016) which is hereby incorporated by reference in its entirety.

What is claimed is:

1. A system, comprising:
at least one processor configured to:
receive a time-series generated by an ultrasonic flow sensor corresponding to a flow of at least one fluid through a fluid flow path of the ultrasonic flow sensor;
generate, using at least one machine learning model, based on the time-series, at least one of (i) a predicted start time associated with a start of the flow of the at least one fluid through the fluid flow path of the ultrasonic flow sensor, (ii) a predicted time period associated with the flow of the at least one fluid through the fluid flow path of the ultrasonic flow sensor, (iii) a predicted end time associated with an end of the flow of the at least one fluid through the fluid flow path of the ultrasonic flow sensor, or any combination thereof; and
provide the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the end predicted time associated with the end of the flow of the at least one fluid through the fluid flow path of the ultrasonic flow sensor, or any combination thereof.

2. The system of claim 1, wherein the time-series includes at least one of a plurality of amplitudes at a plurality of time points, a plurality of phases at the plurality of time points, or any combination thereof, and wherein the at least one processor is further configured to:
generate, using at least one feature extraction algorithm, based on the time-series including the plurality of amplitudes at the plurality of time points, the plurality of phases at the plurality of time points, or any combination thereof, a feature representation associated with the time-series, and
wherein the at least one processor is configured to generate, using the at least one machine learning model, based on the time-series, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, by:
providing, as input to the at least one machine learning model, the feature representation associated with the time-series; and
receiving, as output from the at least one machine learning model, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof.

3. The system of claim 1, wherein the at least one processor is further configured to:
identify, based on the time series generated by the ultrasonic flow sensor corresponding to the flow of the at least one fluid through the fluid flow path of the ultrasonic flow sensor, in a database including a plurality of stored feature representations associated with a plurality of medication types, at least one stored feature representation of the plurality of stored feature representations, wherein the at least one stored feature representation is associated with at least one medication type; and
provide an indication of the at least one medication type.

4. The system of claim 3, wherein the at least one processor is further configured to:
obtain at least one expected medication type;
determine whether the at least one expected medication type corresponds to the at least one medication type associated with the identified at least one stored feature representation; and
provide an indication of whether the at least one expected medication type corresponds to the at least one medication type associated with the identified at least one stored feature representation.

5. The system of claim 4, wherein the at least one processor is configured to generate, using the at least one machine learning model, based on the time-series and at least one of the at least one expected medication type, the at least one medication type, or any combination thereof, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof.

6. The system of claim 1, wherein the at least one processor is further configured to:
determine, based on the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, at least one of an amount of the at least one fluid that flowed through the fluid flow path of the ultrasonic flow sensor, a rate at which the at least one fluid flowed or is flowing through the fluid flow path of the ultrasonic flow sensor, or any combination thereof; and
provide the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof.

7. The system of claim 6, wherein the at least one processor is further configured to:
automatically store, in a database, in association with a record of a patient, the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof.

8. The system of claim 6, wherein the at least one processor is further configured to:
determine whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates at least one of a threshold amount associated with at least one fluid, a threshold rate associated with the at least one fluid, or any combination thereof; and
provide an indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof.

9. The system of claim 8, wherein the at least one processor is configured to provide the indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof, by controlling a user interface associated with the ultrasonic flow sensor to output at least one of an audio output, a visual output, or any combination thereof associated with whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof.

10. The system of claim 1, further comprising:
the ultrasonic flow sensor, wherein the ultrasonic flow sensor includes a flow tube that defines the fluid flow path of the ultrasonic flow sensor, a first piezoelectric sensor or transducer arranged at an upstream position of the flow tube, and a second piezoelectric sensor or transducer arranged at a downstream position of the flow tube, and
wherein the first piezoelectric sensor or transducer and the second piezoelectric sensor or transducer are configured to generate the time-series corresponding to the flow of the at least one fluid through the fluid flow path of the ultrasonic flow sensor.

11. The system of claim 1, wherein the at least one fluid includes a sequence of fluids,
wherein the time-series is generated by the ultrasonic flow sensor corresponding to a sequence of flows of the sequence of fluids through the fluid flow path of the ultrasonic flow sensor, and
wherein the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof includes:
at least one of (a) one or more predicted start times associated with one or more starts of one or more flows of one or more fluids of the sequence of fluids through the fluid flow path of the ultrasonic flow sensor, (b) one or more predicted time periods associated with the one or more flows of the one or more fluids of the sequence of fluids through the fluid flow path of the ultrasonic flow sensor, (c) one or more predicted end times associated with one or more ends of the one or more flows of the one or more fluids of the sequence of fluids through the fluid flow path of the ultrasonic flow sensor, or any combination thereof.

12. The system of claim 11, wherein the time-series is further generated by the ultrasonic flow sensor corresponding to at least one period of no fluid flow through the fluid flow path of the ultrasonic flow sensor, and wherein the at least one processor is further configured to:
generate, using the at least one machine learning model, based on the time-series, at least one of (x) a predicted no flow start time associated with a start of the at least one period of no fluid flow through the fluid flow path of the ultrasonic flow sensor, (y) a predicted no flow time period associated with the at least one period of no fluid flow through the fluid flow path of the ultrasonic flow sensor, (z) a predicted no flow end time associated with an end of the at least one period of no fluid flow through the fluid flow path of the ultrasonic flow sensor, or any combination thereof.

13. The system of claim 12, wherein the at least one period of no fluid flow through the fluid flow path of the ultrasonic sensor occurs before the flow of the fluid of the sequence of fluids through the fluid flow path of the ultrasonic sensor.

14. The system of claim 12, wherein the at least one period of no fluid flow through the fluid flow path of the ultrasonic sensor occurs after a flow of a fluid of the sequence of fluids through the fluid flow path of the ultrasonic sensor and before a flow of a next fluid of the sequence of fluids through the fluid flow path of the ultrasonic flow sensor.

15. The system of claim 12, wherein the sequence of fluids includes at least one saline bolus and at least one medication bolus.

16. A method, comprising:
receiving, with at least one processor, a time-series generated by an ultrasonic flow sensor corresponding to a flow of at least one fluid through a fluid flow path of the ultrasonic flow sensor;
generating, with at least one processor, using at least one machine learning model, based on the time-series, at least one of (i) a predicted start time associated with a start of the flow of the at least one fluid through the fluid flow path of the ultrasonic flow sensor, (ii) a predicted time period associated with the flow of the at least one fluid through the fluid flow path of the ultrasonic flow sensor, (iii) a predicted end time associated with an end of the flow of the at least one fluid through the fluid flow path of the ultrasonic flow sensor, or any combination thereof; and
providing, with at least one processor, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the end predicted time associated with the end of the flow of the at least one fluid through the fluid flow path of the ultrasonic flow sensor, or any combination thereof.

17. The method of claim 16, wherein the time-series includes at least one of a plurality of amplitudes at a plurality of time points, a plurality of phases at the plurality of time points, or any combination thereof, and wherein the method further comprises:
generating, with the at least one processor, using at least one feature extraction algorithm, based on the time-series including the plurality of amplitudes at the plurality of time points, the plurality of phases at the plurality of time points, or any combination thereof, a feature representation associated with the time-series, and
wherein generating, with the at least one processor, using the at least one machine learning model, based on the time-series, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, includes:
providing, with the at least one processor, as input to the at least one machine learning model, the feature representation associated with the time series; and
receiving, with the at least one processor, as output from the at least one machine learning model, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof.

18. The method of claim 16, further comprising:
identifying, with the at least one processor, based on the time series generated by the ultrasonic flow sensor corresponding to the flow of the at least one fluid through the fluid flow path of the ultrasonic flow sensor, in a database including a plurality of stored feature representations associated with a plurality of medication types, at least one stored feature representation of the plurality of stored feature representations, wherein the at least one stored feature representation is associated with at least one medication type; and
providing, with the at least one processor, an indication of the at least one medication type.

19. The method of claim 18, further comprising:
obtaining, with the at least one processor, at least one expected medication type;
determining, with the at least one processor, whether the at least one expected medication type corresponds to the at least one medication type associated with the identified at least one stored feature representation; and
providing, with the at least one processor, an indication of whether the at least one expected medication type corresponds to the at least one medication type associated with the identified at least one stored feature representation.

20. The method of claim 19, wherein generating, with the at least one processor, using the at least one machine learning model, based on the time-series, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, includes:
generating, with the at least one processor, using the at least one machine learning model, based on the time-series and at least one of the at least one expected medication type, the at least one medication type, or any combination thereof, the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof.

21. The method of claim 16, further comprising:
determining, with the at least one processor, based on the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof, at least one of an amount of the at least one fluid that flowed through the fluid flow path of the ultrasonic flow sensor, a rate at which the at least one fluid flowed or is flowing through the fluid flow path of the ultrasonic flow sensor, or any combination thereof; and
providing, with the at least one processor, the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof.

22. The method of claim 21, further comprising:
automatically storing, with the at least one processor, in a database, in association with a record of a patient, the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof.

23. The method of claim 21, further comprising:
determining, with the at least one processor, whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates at least one of a threshold amount associated with at least one fluid, a threshold rate associated with the at least one fluid, or any combination thereof; and
providing, with the at least one processor, an indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof.

24. The method of claim 23, wherein providing, with the at least one processor, the indication of whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof, includes:
controlling a user interface associated with the ultrasonic flow sensor to output at least one of an audio output, a visual output, or any combination thereof associated with whether the at least one of the amount of the at least one fluid that flowed through the fluid flow path of the ultrasonic flow sensor, the rate at which the at least one fluid flowed or is flowing through the fluid flow path of the ultrasonic flow sensor, or any combination thereof, violates the at least one of the threshold amount associated with at least one fluid, the threshold rate associated with the at least one fluid, or any combination thereof.

25. The method of claim 16, further comprising:
generating, with the ultrasonic flow sensor, the time-series corresponding to the flow of the at least one fluid through the fluid flow path of the ultrasonic flow sensor.

26. The method of claim 16, wherein the at least one fluid includes a sequence of fluids, and
wherein the method further comprises:
generating, with the ultrasonic flow sensor, the time-series corresponding to a sequence of flows of the sequence of fluids through the fluid flow path of the ultrasonic flow sensor,
wherein the at least one of (i) the predicted start time associated with the start of the flow of the at least one fluid through the fluid flow path of the ultrasonic flow sensor, (ii) the predicted time period associated with the flow of the at least one fluid through the fluid flow path of the ultrasonic flow sensor, (iii) the predicted end time associated with the end of the flow of the at least one fluid through the fluid flow path of the ultrasonic flow sensor, or any combination thereof includes:
at least one of (a) one or more predicted start times associated with one or more starts of one or more flows of one or more fluids of the sequence of fluids through the fluid flow path of the ultrasonic flow sensor, (b) one or more predicted time periods associated with the one or more flows of the one or more fluids of the sequence of fluids through the fluid flow path of the ultrasonic flow sensor, (c) one or more predicted end times associated with one or more ends of the one or more flows of the one or more fluids of the sequence of fluids through the fluid flow path of the ultrasonic flow sensor, or any combination thereof.

27. The method of claim 26, further comprising:
generating, with the ultrasonic flow senor, the time-series corresponding to at least one period of no fluid flow through the fluid flow path of the ultrasonic flow sensor; and
generating, with the at least one processor, using the at least one machine learning model, based on the time-series, at least one of (x) a predicted no flow start time associated with a start of the at least one period of no fluid flow through the fluid flow path of the ultrasonic flow sensor, (y) a predicted no flow time period associated with the at least one period of no fluid flow through the fluid flow path of the ultrasonic flow sensor, (z) a predicted no flow end time associated with an end of the at least one period of no fluid flow through the fluid flow path of the ultrasonic flow sensor, or any combination thereof.

28. The method of claim 27, wherein the at least one period of no fluid flow through the fluid flow path of the ultrasonic sensor occurs before the flow of the fluid of the sequence of fluids through the fluid flow path of the ultrasonic sensor.

29. The method of claim 27, wherein the at least one period of no fluid flow through the fluid flow path of the ultrasonic sensor occurs after a flow of a fluid of the sequence of fluids through the fluid flow path of the ultrasonic sensor and before a flow of a next fluid of the sequence of fluids through the fluid flow path of the ultrasonic flow sensor.

30. The method of claim 27, wherein the sequence of fluids includes at least one saline bolus and at least one medication bolus.
